(19) United States
(12) Patent Application Publication
(10) Pub. No.: US 2016/0025734 A1
(43) Pub. Date: Jan. 28, 2016

(54) USE OF URINARY PROTEIN BIOMARKERS TO DISTINGUISH BETWEEN NEOPLASTIC AND NON-NEOPLASTIC DISEASE OF THE PROSTATE

(71) Applicants: Children's Medical Center Corporation, Boston, MA (US); The Brigham and Women's Hospital, Inc., Boston, MA (US)

(72) Inventors: Marsha A. Moses, Brookline, MA (US); Kevin Loughlin, Boston, MA (US); Andrej Jedinak, Brighton, MA (US); Adam Curatolo, Princeton, WV (US)

(21) Appl. No.: 14/805,668

(22) Filed: Jul. 22, 2015

Related U.S. Application Data
(60) Provisional application No. 62/027,927, filed on Jul. 23, 2014.

(51) Int. Cl. G01N 33/574

(52) U.S. Cl. CPC G01N 33/57434; G01N 2560/00

(57) ABSTRACT

The disclosure provides methods to facilitate the diagnosis of prostate cancer. In particular, the methods disclosed herein can be used to distinguish prostate cancer from benign prostatic hyperplasia. The methods comprise determining in a sample of the subject a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein, and correlating the level of the one or more biomarkers to a reference level to facilitate diagnosis of prostate cancer or BPH.

### RELATED APPLICATIONS

This application claims the benefit under 35 U.S.C. §119(e) of U.S. Provisional Patent Application filed Jul. 23, 2014, entitled “USE OF URINARY PROTEINS BIOMARKERS TO DISTINGUISH BETWEEN NEOPLASTIC AND NON-NEOPLASTIC DISEASE OF THE PROSTATE”, Ser. No. 62/027,927, the content of which is incorporated by reference in its entirety.

### BACKGROUND OF THE INVENTION

Prostate cancer is the second most frequently diagnosed form of cancer in the world and the sixth most deadly form of male cancer.¹ In 2013, in the United States alone, it is projected that prostate cancer (PCa) will account for 28% (238 590) of cancer cases and 10% (29 720) of cancer deaths². PCa and benign prostate hyperplasia (BPH) are common diseases of the aging male³ and share similar symptoms of frequent urination, nocturia, difficulty starting and maintaining a steady stream of urine, hematuria, dysuria, erectal dysfunction, and painful ejaculation.⁴⁻⁸ Benign prostatic hyperplasia (BPH) is the most common benign disease among men worldwide and its incidence increases with age. A male infant born today has an approximate one in six lifetime risk of developing prostate cancer.⁹ As with BPH, the incidence of prostate cancer increases as men age beyond 40. Prostate specific antigen (PSA) is elaborated by both benign and malignant prostate cells. An elevated serum PSA can be detected with either benign or malignant growth of the prostate. A modest elevation of PSA between 4 and 10 ng/mL confers an approximate 25 percent risk of a positive biopsy for prostate cancer.¹⁰ It is the demographic overlap of BPH and prostate cancer, and the lack of discrimination between these two prostate diseases by PSA, that defines the diagnostic dilemma confronting clinicians who treat prostate disease.

The PSA test has been widely used as a diagnostic, screening, and monitoring tool since it was first approved in 1986 by the US Food and Drug Administration as an aid for the early detection of prostate cancer.¹¹ However, the PSA test lacks high sensitivity and specificity for PCa and PSA levels are frequently elevated in benign conditions, including BPH.¹² This leads to the situation in which men continue to be over-diagnosed and unnecessarily biopsied with indolent disease or BPH.¹³ Side effects of prostate biopsies have been associated with infection, bleeding, urinary difficulty, fever, urinary retention, prostatitis, urosepsis, hematuria, and hematospermia.¹⁴ Post-biopsy infection rates of 8% (even with antibiotic prophylaxis) have been reported.¹⁵ A variety of permutations of PSA have been utilized to attempt to enhance the diagnostic sensitivity and specificity of PSA screening. However, age-adjusted PSA ranges, PSA velocity, PSA density, and free PSA fraction have all been disappointing in their ability to discriminate between BPH and prostate cancer,¹⁶ making it very difficult to differentiate between these two diseases in a clinical setting. Currently, prostate cancer prognosis is based on age, elevated levels of serum prostate specific antigen (PSA), and a prostatic digital rectal examination (DRE) often followed by prostate biopsy.¹⁷

In May 2012, the United States Preventive Services Task Force (USPSTF) announced that PSA screening for prostate cancer demonstrated small potential benefit against a backdrop of potential harms.¹⁸ There is, therefore, an urgent need for novel biomarkers that can distinguish between patients with BPH vs. PCa.

### SUMMARY OF THE INVENTION

The present disclosure, is based at least in part, on the discovery of new biomarkers that can distinguish between BPH and PCa. Accordingly, some aspects of the disclosure provide a method for distinguishing prostate cancer from benign prostatic hyperplasia (BPH) in a subject. The method comprises determining in a sample of the subject a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein, and correlating the level of the one or more biomarkers to a reference level to facilitate diagnosis of prostate cancer or BPH.

In some embodiments, the method further comprises providing a report indicating that the subject has prostate cancer when the level of the one or more biomarkers is increased as compared to a reference level. In some embodiments, the method further comprises providing a report indicating that the subject does not have prostate cancer when the level of the one or more biomarkers is decreased or remains unchanged as compared to a reference level.

In some embodiments, the reference level is the level of the one or more biomarkers in a control subject who does not have prostate cancer. In some embodiments, the control subject has BPH. In some embodiments, the biomarkers comprise MUC3, PGA3, β2M and PIK3IP1. In some embodiments, the biomarker is not β2M. In some embodiments, the sample is selected from the group consisting of blood, serum, urine, prostatic fluid, seminal fluid, semen, and prostate tissue. In some embodiments, the sample is urine. In some embodiments, the level of the one or more biomarkers is determined using mass spectrometry. In some embodiments, the level of the one or more biomarkers is determined using isobaric tags for relative and absolute quantitation (iTRAQ) LC/LC/MS/MS. In some embodiments, the level of the one or more biomarkers is determined by immunoassay. In some embodiments, the level of the one or more biomarkers is determined by an enzyme-linked immunosorbant assay. In some embodiments, the diagnosis of the subject is made in combination with a prostate specific antigen (PSA) test.

Some aspects of the disclosure provide a method for distinguishing prostate cancer from benign prostatic hyperplasia (BPH) in a subject. The method comprises performing an assay to determine a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein in a biological sample obtained of the subject, and providing a report indicating that the subject has prostate cancer when the level of the one or more biomarkers is increased as compared to a reference level or providing a report indicating that the subject does not have prostate cancer when the level of the one or more biomarkers is decreased or remains unchanged as compared to the reference level.

Some aspects of the disclosure provide a method for diagnosing prostate cancer in a subject. The method comprises determining in a biological sample of the subject a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein, wherein an increased level of the one or more biomarkers as compared to a reference level is indicative that the subject has prostate cancer, and wherein a decreased level or an unchanged level of the one or more biomarkers as compared to a reference level is indicative that the subject does not have prostate cancer.

In some embodiments, the method further comprises providing a report indicating that the subject has prostate cancer when the level of the one or more biomarkers is increased as compared to a reference level. In some embodiments, the method further comprises providing a report indicating that the subject does not have prostate cancer when the level of the one or more biomarkers is decreased or remains unchanged as compared to a reference level.

In some embodiments, the reference level is the level of the one or more biomarkers in a control subject who does not have prostate cancer. In some embodiments, the control subject has BPH. In some embodiments, the biomarkers comprise MUC3, PGA3, β2M and PIK3IP1. In some embodiments, the biomarker is not β2M. In some embodiments, the sample is selected from the group consisting of blood, serum, urine, prostatic fluid, seminal fluid, semen, and prostate tissue. In some embodiments, the sample is urine. In some embodiments, the level of the one or more biomarkers is determined using mass spectrometry. In some embodiments, the level of the one or more biomarkers is determined using isobaric tags for relative and absolute quantitation (iTRAQ) LC/LC/MS/MS. In some embodiments, the level of the one or more biomarkers is determined by immunoassay. In some embodiments, the level of the one or more biomarkers is determined by an enzyme-linked immunosorbant assay. In some embodiments, the diagnosis of the subject is made in combination with a prostate specific antigen (PSA) test.

Some aspects of the disclosure involve a method for monitoring prostate cancer recurrence in a post-prostate cancer treatment subject. The method comprises determining a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein in a first biological sample of a post-prostate cancer treatment subject, determining a level of the one or more biomarkers in a second biological sample of the post-prostate cancer treatment subject, comparing the level of the one or more biomarkers in the first and second biological samples, and referring the post-prostate cancer treatment subject for treatment of recurrence of prostate cancer when the level of the one or more biomarkers in the second biological sample is increased as compared to the level of the one or more biomarkers in the first biological sample.

In some embodiments, the method further comprises providing a report indicating that the subject has recurrence of prostate cancer when the level of the one or more biomarkers in the second biological sample is greater than the level of the one or more biomarkers in the first biological sample. In some embodiments, the method further comprises providing a report indicating that the subject does not have recurrence of prostate cancer when the level of the one or more biomarkers in the second biological sample is decreased or remains unchanged as compared to the level of the one or more biomarkers in the first biological sample.

In some embodiments, the biomarkers comprise MUC3, PGA3, β2M and PIK3IP1. In some embodiments, the biomarker is not β2M. In some embodiments the sample is selected from the group consisting of blood, serum, urine, prostatic fluid, seminal fluid, semen, and prostate tissue. In some embodiments, the sample is urine. In some embodiments, the level of the one or more biomarkers is determined using mass spectrometry. In some embodiments, the level of the one or more biomarkers is determined using isobaric tags for relative and absolute quantitation (iTRAQ) LC/LC/MS/MS. In some embodiments, the level of the one or more biomarkers is determined by immunoassay. In some embodiments, the level of the one or more biomarkers is determined by an enzyme-linked immunosorbant assay. In some embodiments, the method further comprises monitoring prostate specific antigen (PSA) levels of the post-prostate cancer treatment subject.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows urinary proteins significantly differentially expressed between BPH vs. PCa identified by iTRAQ. The relative level of protein expression is shown with a pseudo color scale (−3 to 3), with red denoting up-regulation and green denoting down-regulation. The columns represent samples and the rows represent the proteins. From top to bottom the proteins are: (1) OPN-c [Homo sapiens]; (2) unnamed protein product [Homo sapiens]; (3) intestinal mucin [Homo sapiens]; (4) “uromodulin (uromucoid, Tamm-Horsfall glycoprotein), isoform CRA_a [Homo sapiens]”; (5) HGFL protein [Homo sapiens]; (6) epidermal growth factor (beta-urogastrone) [Homo sapiens]; (7) prion protein [Homo sapiens]; (8) kininogen 1 variant [Homo sapiens]; (9) cadherin 13 preproprotein [Homo sapiens]; (10) ankyrin repeat domain 11 [Homo sapiens]; (11) Collagen alpha 1 chain precursor variant [Homo sapiens]; (12) WAP four-disulfide core domain 2 precursor [Homo sapiens]; (13) Collagen alpha-1(III) chain precursor [Homo sapiens]; (14) pro-alpha 2(I) collagen [Homo sapiens]; (15) sulfatase 2 isoform b precursor [Homo sapiens]; (16) apolipoprotein D; apoD [Homo sapiens]; (17) MASP-2 protein [Homo sapiens]; (18) “pepsinogen 3, group I [Homo sapiens]”; (19) alpha-1-acid glycoprotein 1 precursor [Homo sapiens]; (20) “alpha-2-glycoprotein 1, zinc [Homo sapiens]”; (21) “inositol 1,4,5-triphosphate receptor, type 2, isoform CRA_b [Homo sapiens]”; (22) unnamed protein product [Homo sapiens]; (23) alpha2-HS glycoproteins [Homo sapiens]; (24) “HSCARG protein, isoform CRA_b [Homo sapiens]”; (25) B2M [Homo sapiens]; and (26) SNC66 protein [Homo sapiens].

FIG. 2 shows the immunoblot analyses of urine samples from BPH and prostate cancer patients. Representative urine samples were separated on 4-12% Bis-Tris gels under reducing conditions and were subsequently subjected to western blot analysis using the appropriate antibody for the following iTRAQ-identified proteins: mucin 3 fragment (MUC3 25 kDa), mucin 3 fragment (MUC3 51 kDa), β-2-microglobulin (β2M), pepsinogen 3 (PGA3), apoliprotein D (ApoD), alpha-2-glycoprotein 1, zinc (ZAG) and uromodulin (THP). One hundred seventy-three (173) urine samples from patients diagnosed with benign disease (N=83) and tumor disease (N=90) were analyzed.

FIG. 3 shows the probability of PCa according to urinary biomarkers stratified by PSA level: A) Probability of PCa according to β2M stratified by PSA level, β2M≧40 DU (P<0.001) B) Probability of PCa according to β2M stratified by PSA level, PGA3≧190 DU (P=0.008) B) Probability of PCa according to PGA3 stratified by PSA level C) Probability of PCa according to MUC3 stratified by PSA level, MUC3≧185 DU (P=0.009).

FIG. 4 shows the pathways and functional enrichment analysis of proteins differentially expressed in prostate cancer as compared to BPH: A) Functional enrichment analysis and B) Pathways Enrichment analysis. Each bar represents a significantly enriched pathway or function as determined by the multiple tests corrected Fisher's Exact Test P value. The P value is depicted as −log 10 (B-H P value) on primary X-axis. The analysis for canonical pathways and functions was performed using Ingenuity Pathway Analysis. From top to bottom the GO Categories of A) are: (1) DNA Replication, Recombination, and Repair; (2) Drug Metabolism; (3) Cell Death; (4) Cellular Growth and Proliferation; (5) Cellular Development; (6) Cell Cycle; (7) Anigen Presentation; (8) Small Molecule Biochemistry; (9) Carbohydrate Metabolism; (10) Cell Morphology; (11) Vitamin and Mineral Metabolism; (12) Molecular Transport; (13) Cell Signaling; (14) Cell-to-Cell Signaling and Interaction; (15) Cellular Movement; (16) Cellular Function and Maintenance; and (17) Cellular Assembly and Organization. From top to bottom the Pathways of B) are: (1) Clathrin-medicated Endocytosis Signaling; (2) Production of Nitric Oxide and Reactive Oxygen Species in Macrophages; (3) IL-12 Signaling and Production in Macrophages; (4) eNOS Signaling; (5) Communication between Innate and Adaptive Immune Cells; (6) Nitric Oxide Signaling in the Cardiovascular System; (7) Dendritic Cell Maturation; (8) LXR/RXR Activation; and (9) Intrinsic Prothrombin Activation Pathway.

FIG. 5 is an interactive network of the proteins that are differentially expressed in prostate cancer as compared to BPH. The network node and edges represent proteins and their interactions respectively. The intensity of the node color indicates the degree of up-regulation (red) or down-regulation (green), while white nodes indicate non-modified proteins that may be affected by post-translational modification. All networks shown were significantly affected in prostate cancer, with a score >15. The network analysis identified many focus hubs (e.g., NFκB, ERK1/2, Collagen, TGFβ, PI3K, p38 MAPK) with high degree of interactions.

FIG. 6 show receiver operating characteristic curves for combined urinary biomarkers in differentiating BPH patients from PCa patients. White circles represent the ROC curve (AUC=0.734) for three clinically relevant PSA categories (0-4, 4.1-10, >10 ng/mL). Black triangles signify the ROC curve based on the combination of three urinary biomarkers with PSA categories and demonstrate the highest diagnostic accuracy (AUC=0.812), representing significant improvement (P=0.004).

FIG. 7 shows the probability of PCa according to PIK3IP1 stratified by PSA level.

FIG. 8 shows protein blots of metastatic prostate cancer urine samples.

### DETAILED DESCRIPTION OF THE INVENTION

Currently, clinicians cannot discriminate between benign prostatic hyperplasia (BPH) and prostate cancer (PCa). This disclosure, is based at least in part, on the discovery of new biomarkers that can distinguish between BPH and PCa. Accordingly, aspects of the disclosure include methods for distinguishing prostate cancer from benign prostatic hyperplasia, methods for diagnosing prostate cancer and methods for monitoring for prostate cancer recurrence.

As used herein, “prostate cancer” refers to any cancer of the prostate gland in which cells of the prostate mutate and begin to multiply out of control. The term “prostate cancer” includes early stage, localized cancer of the prostate gland; later stage, locally advanced cancer of the prostate gland; and later stage metastatic cancer of the prostate gland (in which the cancer cells spread (metastasize) from the prostate to other parts of the body, especially the bones and lymph nodes). Prostate cancer can include adenocarcinoma, leiomyosarcoma, rhabdomyosarcoma, intraepithelial neoplasia, or sarcoma. Prostate cancers can be encapsulated or metastatic.

As used herein “benign prostatic hyperplasia” or hypertrophy” (BPH) refers to a disease or condition of the prostate gland wherein the prostate is enlarged or exhibits hyperplasia. BPH is not prostate cancer. BPH is a common urological condition caused by the non-cancerous enlargement of the prostate gland in aging men. BPH affects the inner part of the prostate first—the part that encircles the urethra as it leaves the bladder. (The urethra is the tube that carries urine and semen through the penis and out of the body). As the prostate grows, it may begin to squeeze the upper part of the urethra and restrict the flow of urine. This can cause men to have trouble urinating leading to the symptoms of BPH.

Traditionally, malignant disease is ruled out by performing a prostate-specific antigen (PSA) blood test. However, the subject of PSA screening to detect prostate cancer continues to engender significant controversy. In May 2012, the United States Preventive Services Task Force (USPSTF) announced that PSA screening for prostate cancer demonstrated small potential benefit and exposed patients to potential harms. The American Urological Association, however, responded with its own updated guidelines that, in properly selected patients, PSA screening is beneficial and can save lives.²⁷ The underpinning of this honest disagreement is that PSA is produced by both benign and malignant prostate tissue and no PSA value can rule out the possibility of prostate malignancy, as Thompson and colleagues reported on 2950 men who were part of the Prostate Cancer Prevention study.²⁸ The men in the study ranged in age from 62 to 91 years, and prostate cancer was diagnosed in 449 of them (15.2%). However, prostate cancer was diagnosed even in those men who had very low PSA levels. The prevalence of prostate cancer was 6.6 percent among men with a PSA level of up to 0.5 ng/ml, 10.1 percent among those with values of 0.6 to 1.0 ng/ml, 17 percent among those with values of 1.1 to 2.0 ng/ml, 23.9 percent among those with values of 2.1 to 3.0 ng/ml, and 26.9 percent among those with values of 3.1 to 4.0 ng/ml. The authors' overall findings were that as many as 15 percent of men with so-called “normal” PSA levels did, in fact, have prostate cancer. Thus, this study demonstrated the unreliability of PSA testing alone to diagnose prostate cancer and underscored the need for new biomarkers with increased sensitivity and specificity for prostate cancer detection.

The biomarkers described herein were able to independently discriminate between BPH and early stages of PCa. In addition, when these markers are multiplexed, the accuracy in differentiating between BPH and early PCa is further increased. Importantly, the described panel of biomarkers, when multiplexed with clinically defined categories of PSA, effectively distinguishes BPH from early PCa with high sensitivity and specificity.

Accordingly, some aspects of the disclosure involve methods for distinguishing prostate cancer from benign prostatic hyperplasia (BPH) in a subject. The methods comprise determining in a sample of the subject a level of one or more biomarkers selected from the group consisting of: mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein, and correlating the level of the one or more biomarkers to a reference level to facilitate diagnosis of prostate cancer or BPH.

Some aspects of the disclosure involve methods for diagnosing prostate cancer in a subject. The method comprises determining in a biological sample of the subject a level of one or more biomarkers selected from the group consisting of: mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein, wherein an increased level of the one or more biomarkers as compared to a reference level is indicative that the subject has prostate cancer and wherein a decreased or an unchanged level of the one or more biomarkers is indicative that the subject has BPH. In some embodiments, a decreased or an unchanged level of the one or more biomarkers is indicative that the subject does not have prostate cancer.

Some aspects of the disclosure involve methods for monitoring prostate cancer recurrence in a post-prostate cancer treatment subject. The method comprises determining a level of one or more biomarkers selected from the group consisting of: mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein in a first biological sample of a post-prostate cancer treatment subject, determining a level of the one or more biomarkers in a second biological sample of the post-prostate cancer treatment subject, comparing the level of the one or more biomarkers in the first and second biological samples, and referring the post-prostate cancer treatment subject for treatment of recurrence of prostate cancer when the level of the one or more biomarkers in the second biological sample is higher than the level of the one or more biomarkers in the first biological sample.

A protein found to be significantly elevated in urine of PCa patients was intestinal mucin 3 (MUC3; GI 2853295), a member of the membrane-associated mucins which may be shed from the cell surface via activation of membrane-associated metalloproteinases.³¹⁻³³ MUC3 is a large glycoprotein expressed by the human intestine and gall bladder. Previous studies showed a correlation between elevated MUC3 expression and esophageal,³⁴ gastric,³⁵ breast,³⁶ and colon cancer.³⁷ MUC3 was found able to differentiate between BPH and early PCa. In addition, the ability of MUC3 to discriminate between BPH and early PCa was strengthened when MUC3 was multiplexed with clinically defined categories of PSA, making it a prospective biomarker for differentiating BPH from early PCa.

Pepsinogen 3, group 1 (PGA3; GI 119372298; Pepsin A-3 preproprotein) was also found to be elevated in the urine of PCa patients but not in BPH. PGA3 encodes human pepsinogen A enzyme, which is synthesized and secreted by the gastric chief cells of the human stomach before being converted into the proteolytic enzyme pepsin A, an upstream step in the digestive process.³⁸ Low levels of PGA in serum³⁹ as well as decreased or lost expression of PGA in gastric tissue and cancer cell lines were previously reported.⁴⁰ In contrast, a recent study demonstrated increased mRNA levels of PGA in seven colorectal cancer cell lines.⁴¹ The present study shows that PGA3 can be used to distinguish between patients with BPH or with early-stage PCa.

β2 microglobulin (β2M; GI 48146249), a component of the major histocompatibility complex class I (MHC I), was the third protein identified via iTRAQ and validated by immunoblot analysis. Increased expression of β2M has been previously associated with breast,⁴² renal,⁴³ lung,⁴⁴ colon,⁴⁵ and hematologic malignancies.⁴⁶ β2M levels were also significantly elevated in urine⁴⁷ and in serum⁴⁸ of prostate cancer patients compared to healthy subjects. The present study demonstrates that β2M effectively discriminates between BPH and early stages of PCa.

Uromodulin (uromucoid, Tamm-Horsfall glycoprotein), isoform CRA_a (GI 119570699) is known also as Tamm-Horsfall glycoprotein or THP. It is a glycoprotein that in humans is encoded by the UMOD gene. Uromodulin is the most abundant protein excreted in ordinary urine. It functions in biogenesis and organization of the apical membrane of epithelial cells of the thick ascending limb of Henle's loop (TALH), where it promotes formation of complex filamentous gel-like structure providing the water barrier permeability.

Prion protein (GI 119490000) is encoded by the PRNP gene. It's proposed function include the transport of copper into cells and protection of brain cells (neurons) from injury (neuroprotection). Studies have also suggested a role for PrP in the formation of synapses, which are the junctions between nerve cells (neurons) where cell-to-cell communication occurs.

HGFL (phosphoinositide-3-kinase-interacting protein 1 isoform 1 precursor; GI 51317358) is phosphatidylinositol-3-kinases (PI3K) interacting protein 1, known as PIK3IP1, which plays role in PI3K/Akt signaling and is dysregulated in 70% of prostate cancers. PIK3IP1 and PI3K subunit p85 β reaches 70% homology in sequence. PIK3IP1 directly binds to the p110 catalytic subunit and down-modulates PI3K activity. It had been shown that PIK3IP1 suppresses the development of hepatocellular carcinoma and plays an inhibitory role in T-cell activation.

Apolipoprotein D (GI 1246096) is known also as Apo D is a component of high density lipoprotein. It is a glycoprotein of estimated molecular weight 33 KDa. Apo D occurs in the macromolecular complex with lecithin-cholesterol acyltransferase. It is assumed to be involved in the transport and binding of bilin.

WAP four-disulfide core domain protein 2 (GI 56699495; WFDC2) is also known as Human Epididymis Protein 4 HE4). It is a protein that in humans is encoded by the WFDC2 gene. It is expressed in a number of normal tissues, including male reproductive system, regions of the respiratory tract and nasopharynx. WFDC2 is highly expressed in a number of tumors cells lines, such ovarian, colon, breast, lung and renal cells lines.

Kininogen 1 variant (GI 62898910; KNG1) is also known as alpha-2-thiol proteinase inhibitor, Williams-Fitzgerald-Flaujeac factor or the HMWK-kallikrein factor. It is a protein that in humans is encoded by the KNG1 gene. Kininogen-1 is essential for blood coagulation and assembly of the kallikrein-kinin system.

Collagen alpha-1(III) chain (GI 124056490; COL3A1) is a protein that in humans is encoded by the COL3A1 gene, which is located on chromosome 2. Collagen type III occurs in most soft connective tissues along with type I collagen. Involved in regulation of cortical development. Is the major ligand of GPR56 in the developing brain and binding to GPR56 inhibits neuronal migration and activates the RhoA pathway by coupling GPR56 to GNA13 and possibly GNA12.

Osteopontin-c (OPN-c; GI 992950) is a cancer specific splice variant of osteopontin. Osteopontin (OPN), also known as bone sialoprotein I (BSP-1 or BNSP). It's function is linked to cell-matrix interaction. OPN also acts as a cytokine involved in enhancing production of interferon-gamma and interleukin-12 and reducing production of interleukin-10 and is essential in the pathway that leads to type I immunity.

Epidermal growth factor (beta-urogastrone) (GI 4503491) is a 6 kD polypeptide growth factor initially discovered in mouse submaxillary glands. Human epidermal growth factor was originally isolated from urine based on its ability to inhibit gastric secretion and called urogastrone. Epidermal growth factor (EGF) exerts a wide variety of biological effects including the promotion of proliferation and differentiation of mesenchymal and epithelial cells.

Another protein found to be significantly elevated in urine of PCa patients was unnamed protein product (GI 158261423).

Cadherin-13 isoform 1 preproprotein (GI 4502719) is an isoform of cadherin 13 produced by alternative splicing. Cadherin 13 is encoded by CDH13 gene. The encoded protein is localized to the surface of the cell membrane and is anchored by a GPI moiety, rather than by a transmembrane domain. The protein lacks the cytoplasmic domain characteristic of other cadherins, and so is not thought to be a cell-cell adhesion glycoprotein. This protein acts as a negative regulator of axon growth during neural differentiation. It also protects vascular endothelial cells from apoptosis due to oxidative stress, and is associated with resistance to atherosclerosis.

Collagen alpha 1 chain precursor variant (GI 62088774; COL1A1) is encoded by COL1A1 gene. It is also known as alpha-1 type I collagen. COL1A1 encodes the major component of type I collagen, the fibrillar collagen found in most connective tissues, including cartilage.

Ankyrin repeat domain-containing protein 11 (GI 56676397; ANKRD11) is encoded by ANKRD11 gene. NKRD11 is a member of a family of ankyrin repeat-containing cofactors that interacts with p160 nuclear receptor coactivators and inhibits ligand-dependent transcriptional activation. Mutations in this gene have been associated with KBG syndrome, which is characterized by macrodontia, distinctive craniofacial features, short stature, skeletal anomalies, global developmental delay, seizures and intellectual disability.

Pro-alpha 2(I) collagen (GI 2735715) is known also as a collagen, type I, alpha 2 (COL1A2) and is encoded by COL1A2 gene. The COL1A2 gene produces a component of type I collagen called the pro-α2 (I) chain. Type I collagen is the most abundant form of collagen in the human body. Mutations in this gene are associated with osteogenesis imperfecta types I-IV, Ehlers-Danlos syndrome type VIIB, recessive Ehlers-Danlos syndrome Classical type, idiopathic osteoporosis, and atypical Marfan syndrome.

Sulfatase 2 isoform b precursor (GI 38327658; SUMF2) is an enzyme that in humans is encoded by the SUMF2 gene. SUMF2 is also known as pFGE or PSEC0171. It is a 301 amino acid protein that belongs to the sulfatase-modifying factor family and is expressed in lung, heart, placenta, brain, liver, pancreas, skeletal muscle and kidney.

MASP-2 protein (GI 5459324) or Mannan-binding lectin serine protease 2 also known as mannose-binding protein-associated serine protease 2 (MASP-2) is an enzyme that in humans is encoded by the MASP2 gene. MASP2 plays an important role in the activation of the complement system via mannose-binding lectin. Diseases associated with MASP2 include masp2 deficiency, and rheumatic fever.

Inositol 1,4,5-triphosphate receptor, type 2, isoform CRA_b (GI 119616941) is an isoform of inositol 1,4,5-triphosphate receptor, type 2 (ITPR2) produced by alternative splicing. It is in humans is encoded by the ITPR2 gene. Diseases associated with ITPR2 include rheumatic disease, and sjogren's syndrome.

Another protein found to be significantly elevated in urine of PCa patients unnamed protein product (GI 47077082).

Alpha-1-acid glycoprotein 1 precursor (GI 1197209) functions as transport protein in the blood stream. Binds various ligands in the interior of its beta-barrel domain. Also binds synthetic drugs and influences their distribution and availability in the body. Appears to function in modulating the activity of the immune system during the acute-phase reaction. It is well expressed in the liver and is secreted in plasma.

Zinc-alpha-2-glycoprotein precursor (ZAG; GI 4502337) is a protein-coding gene. Diseases associated with AZGP1 include clear cell hidradenoma, and hidrocystoma. Stimulates lipid degradation in adipocytes and causes the extensive fat losses associated with some advanced cancers. May bind polyunsaturated fatty acids.

HSCARG protein, isoform CRA_b (GI 119605708) also known as NMRAL1 (NmrA-like family domain-containing protein 1), is a 299 amino acid redox sensor protein that belongs to the NmrA-type oxidoreductase family. The gene encoding HSCARG maps to human chromosome 16p13.3 and mouse chromosome 16 A1. Overexpression of gene encoding HSCARG gene results in increased viability.

Alpha2-HS glycoprotein (GI 2521983; AHSG) also known as fetuin-A is a protein that in humans is encoded by the AHSG gene. Alpha2-HS glycoprotein, a glycoprotein present in the serum, is synthesized by hepatocytes. The AHSG molecule consists of two polypeptide chains, which are both cleaved from a proprotein encoded from a single mRNA. It is involved in several functions, such as endocytosis, brain development and the formation of bone tissue.

SNC66 protein (GI 17224464) is a Ig-like gene that is down-regulated in colorectal cancer.

In some embodiments, at least some of the biomarkers used in the methods described herein are increased in subject with PCa versus BPH. In some embodiments, the biomarkers that are increased in subjects PCa versus BPH are used in the methods described herein. By “increased” it means that the average expression of a biomarker in a subject with PCa has a statistically significant increase from that in subjects with BPH. For example, a statistically significant increase may be detected when the expression level of the biomarker in a sample of a subject with PCa is at least 1%, at least 5%, at least 10%, at least 25%, at least 50%, at least 100%, at least 250%, at least 500%, or at least 1000% higher than that of a subject with BPH. Similarly, a statistically significant increase may be detected when the expression level of a biomarker in a sample of a subject with PCa is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher than that of a subject with BPH. Statistically significant increases may be identified by using an appropriate statistical test. Tests for statistical significance are well known in the art and are exemplified in Applied Statistics for Engineers and Scientists by Petruccelli, Chen and Nandram 1999 Reprint Ed. In some embodiments, the differentially expressed biomarkers are selected using a criteria of false discovery rate <0.2.

The methods disclosed herein typically comprise determining a level of one or more biomarkers described herein. In some embodiments, a level of a single biomarker is used to facilitate diagnosis of prostate cancer. In some embodiments, a single biomarker is selected from the group consisting of mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein. In some embodiments, the single biomarker is MUC3, PGA3, PIK3IP1 or β2M. In some embodiments, a level of more than one biomarker is used to facilitate diagnosis of prostate cancer. In some embodiments, at least two (2) biomarkers are used. In some embodiments, the at least two biomarkers are selected from the group consisting of mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein. In some embodiments, the at least two biomarkers are MUC3 and PGA3, MUC3 and β2M, MUC3 and PIK3IP1, PGA3 and β2M, PGA3 and PIK3IP1 or β2M and PIK3IP1. In some embodiments, at least three (3) biomarkers are used. In some embodiments, the at least three biomarkers are selected from the group consisting of mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein. In some embodiments, the at least three biomarkers are selected from the group consisting of MUC3, PGA3, PIK3IP1 and β2M. In some embodiments, at least four (4) biomarkers are used. In some embodiments, the at least four biomarkers are selected from the group consisting of mucin 3 (MUC3); pepsinogen 3 preproprotein (PGA3); β-2-microglobulin (β2M); uromodulin; prion protein; PIK3IP1; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein. In some embodiments, the at least four biomarkers are MUC3, PGA3, PIK3IP1 and β2M. In some embodiments, at least 5, at least 10, at least 15, at least 20, or at least 25 of the biomarkers described herein are used in the methods disclosed herein. In some embodiments, all biomarkers discussed herein are used in the methods disclosed herein.

As used herein, a “subject” refers to any male mammal, including humans and non-humans, such as primates. Typically the subject is a human, and is suspected of having prostate cancer. A subject suspected of having PCa may be a subject having one or more clinical symptoms of PCa. A variety of clinical symptoms of prostate cancer are known in the art. Examples of such symptoms include, but are not limited to, frequent urination, nocturia (increased urination at night), difficulty starting and maintaining a steady stream of urine, hematuria (blood in the urine), dysuria (painful urination) and bone pain.

In some embodiments, the subject is a post-prostate cancer treatment subject. A post-prostate cancer treatment subject is a subject who was previously diagnosed and treated for prostate cancer. However, a fraction of the subjects fail the treatment and re-develop prostate cancer. The ability to identify recurrence of the prostate cancer in a post-prostate cancer treatment subject is, therefore, important. Thus, in some embodiments, a post-prostate cancer treatment subject is a subject who was previously treated for prostate cancer and identified as prostate cancer-free after treatment, but who needs to be monitored for recurrence of prostate cancer. In some embodiments, the post-prostate cancer treatment subject has been treated for prostate cancer using radiation therapy, surgery, hormone therapy, chemotherapy, vaccine treatment, cryosurgery, and/or bone directed treatment.

Prostate cancer recurrence is defined as the return of prostate cancer after treatment and after a period of time during which the prostate cancer cannot be detected. (The length of time is not clearly defined). By way of example, the recurrence of prostate cancer may be at the same location (even if the gland was removed) while metastasis is frequently to a different location or tissue, such as to a lymph node or bone as non-limiting examples. In either case it's a prostate cancer recurrence.

The term “sample” or “biological sample” refers to a sample derived from a subject, e.g., a patient. Non-limiting examples of the sample include blood, serum, urine, prostatic fluid, seminal fluid, semen, and prostate tissue. In some embodiments, the sample is urine. With the advantage of being in direct continuity with the prostatic lumen, urine represents a body fluid that is enriched with proteins from PCa cells.¹⁷ In addition, urine-based tests are truly noninvasive¹⁹ and urine is more easily accessible than blood. Thus, in some embodiments, noninvasive urine tests utilizing the biomarkers described herein, in conjunction with clinically defined categories of PSA, can resolve the diagnostic dilemma of effectively discriminating between BPH and early PCa in the clinic.

In some embodiments, multiple samples of the subject are collected over a period of time to monitor (i.e., to detect) recurrence of prostate cancer in a post-prostate cancer treatment subject. As used herein, a “period of time” is intended to include a period of days, weeks, months or even years. Multiple samples of the subject are obtained over a period of time, i.e. a sample is obtained periodically over time at various intervals. A sample can be obtained at any interval. For example, a sample can be taken every day for weeks, months or years. Alternatively, a sample can be obtained once a week, or six times a week for a period of weeks, months or years. In one embodiment, a sample is obtained once a week over a period of three months. In one embodiment, a sample is obtained once a month for a period of months, or years.

Obtaining a sample of a subject means taking possession of a sample of the subject. Obtaining a sample from a subject means removing a sample from the subject. Therefore, the person obtaining a sample of a subject and determining a level of one or more biomarkers in the sample does not necessarily obtain the biological sample from the subject. In some embodiments, the sample may be removed from the subject by a medical practitioner (e.g., a doctor, nurse, or a clinical laboratory practitioner), and then provided to the person determining a level of one or more biomarkers. The sample may be provided to the person determining a level of one or more biomarkers by the subject or by a medical practitioner (e.g., a doctor, nurse, or a clinical laboratory practitioner). In some embodiments, the person determining a level of one or more biomarkers obtains a biological sample from the subject by removing the sample from the subject.

It is to be understood that sample may be processed in any appropriate manner to facilitate measuring a level of the biomarkers. For example, biochemical, mechanical and/or thermal processing methods may be appropriately used to isolate a biomolecule of interest from a biological sample. The level of the biomarkers may also be determined in a sample directly.

As used herein, “determining a level of a biomarker” refers to determining the amount or concentration of the biomarker in the sample. “Determining” may refer to ascertaining, calculating, computing, measuring, perceiving and/or a combination thereof the level of one or more biomarkers. In some embodiments, determining refers to performing an assay to measure the level of one or more biomarkers. In some embodiments, “determining” includes, for example, determining the expression level or activity level of the biomarkers in the sample. In some embodiments, the expression level of the mRNA encoded by the biomarker gene (or a cDNA reverse transcribed therefrom) is determined. In some embodiments, the expression level of the protein encoded by the biomarker gene is determined. In some embodiments, the expression level of the mRNA encoded by the biomarker gene and the expression level of the protein encoded by the biomarker gene is determined.

The level of the biomarkers may be measured by performing an assay. “Performing an assay” means testing a sample to quantify a level of one or more biomarkers described herein. Examples of assays used include, but are not limited to, mass spectroscopy, gas chromatography (GC-MS), HPLC liquid chromatography (LC-MS), immunoassays, Northern blots, and Reverse transcription polymerase chain reaction (RT-PCR). Other appropriate methods for determining a level of biomarkers will be apparent to the skilled artisan.

### Mass Spectrometry

The level of one or more biomarkers may be determined using Mass Spectrometry such as MALDI/TOF (time-of-flight), SELDI/TOF, liquid chromatography-mass spectrometry (LC-MS), iTRAQ LC/LC/MS/MS, gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). see, e.g., U.S. Publication Nos. 20030199001, 20030134304, and 20030077616.

Mass spectrometry methods are well known in the art and have been used to quantify and/or identify proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. Nos. 5,118,937 and 5,045,694.

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to be mixed with the known control sample in the same mass spectrometry run.

In some embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum. The relative signal strengths are an indication of the amount of the biomarker(s) (e.g., MUC3, PGA3, β2M) that is present in the first and second samples. A standard containing a known amount of a biomarker can be analyzed as the second sample to better quantify the amount of the biomarker present in the first sample. In certain embodiments, the identity of the biomarkers in the first and second sample can also be determined. In some embodiments, a biomarker level is measured by MALDI-TOF mass spectrometry.

### Immunoassays

“Radioimmunoassay” is a technique for detecting and measuring the concentration of an antigen using a labeled (e.g., radioactively labeled) form of the antigen. Examples of radioactive labels for antigens include ³H, ¹⁴C, and ¹²⁵I. The concentration of one or more biomarker antigen in a biological sample is measured by having the antigen in the biological sample compete with the labeled (e.g., radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem. 22:895-904.

In a “sandwich ELISA”, an antibody (e.g., anti-MUC3, anti-PGA3, and/or anti-β2M) is linked to a solid phase (i.e., a microtiter plate) and exposed to a biological sample containing antigen (e.g., MUC3, PGA3, and/or β2M). The solid phase is then washed to remove unbound antigen. A labeled antibody (e.g., enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be measured.

In a “competitive ELISA”, antibody (e.g., anti-MUC3, anti-PGA3, and/or anti-β2M) is incubated with a sample containing antigen (i.e., MUC3, PGA3, and/or β2M). The antigen-antibody mixture is then contacted with a solid phase (e.g., a microtiter plate) that is coated with antigen (i.e., MUC3, PGA3, and/or β2M). The more antigen present in the sample, the less free antibody that will be available to bind to the solid phase. A labeled (e.g., enzyme linked) secondary antibody is then added to the solid phase to determine the amount of primary antibody bound to the solid phase.

In a “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or β-galactosidase), or chemical methods (e.g., DAB/Substrate chromagen).

Other techniques may be used to detect the biomarkers of the disclosure, according to a practitioner's preference, and based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that preferentially bind to one or more biomarkers described herein (e.g., anti-MUC3, anti-PGA3, and/or anti-β2M) can then be used to assess MUC3, PGA3, and/or β2M levels, where the intensity of the signal from the detectable label corresponds to the amount of MUC3, PGA3, and/or β2M present. Levels can be quantitated, for example by densitometry.

### RNA Detection Techniques

Detection of RNA transcripts may be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using known amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

In some embodiments, the methods described herein comprise correlating the level of the one or more biomarkers to facilitate diagnosis of prostate cancer or BPH. As used herein, “correlating the level of the one or more biomarkers to a reference level” means comparing the level of the one or more biomarkers to the appropriate reference level. An increase in the level of the one or more biomarkers as compared to the reference level indicates that the subject has prostate cancer. A decrease or no change in the level of the one or more biomarkers as compared to a reference level indicates that the subject does not have prostate cancer. In some embodiments, a decrease or no change in the level of the one or more biomarkers as compared to a reference level indicates that the subject has BPH.

An appropriate reference level of a biomarker can be determined or can be a pre-existing level. An appropriate reference level includes the level of the one or more biomarkers in control subjects who do not have prostate cancer. In some embodiments, the control subjects are normal, healthy subjects. In some embodiments, the control subjects have BPH. In some embodiments, the control subjects are diagnosed as having BPH using one or more of the following tests: American Urological Association (AUA) BPH Symptom Score Index, digital rectal exam (DRE), presence of tumor markers such as PSA, urinary cytology, a measurement of post-void residual volume (PVR) (the amount of urine left in the bladder after urinating), uroflowmetry, or urine flow study (a measure of how fast urine flows when a man urinates), cystoscopy (a direct look in the urethra and/or bladder using a small flexible scope), urodynamic pressure (flow study that tests the pressures inside the bladder during urination) and/or ultrasound of the kidney or the prostate.

In some embodiments, the methods disclosed herein facilitate the diagnosis of prostate cancer. In some embodiments, “facilitate the diagnosis” includes comparing the level of the one or more biomarkers to their appropriate reference level. An increase in the level of the one or more biomarkers as compared to the reference level indicates that the subject has prostate cancer. A decrease or no change in the level of the one or more biomarkers as compared to a reference level indicates that the subject does not have prostate cancer. In some embodiments, a decrease or no change in the level of one or more biomarkers as compared to a reference level indicates that the subject has BPH.

In some embodiments, the methods disclosed herein are performed in combination with other methods known in the art for diagnosing prostate cancer. The methods described herein may be performed in combination with other known methods such as, but not limited to digital rectal exam (DRE), prostate imaging, biopsy with Gleason grading evaluation, presence of tumor markers such as PSA and/or prostate cancer staging (Lumen et al. Screening and early diagnosis of prostate cancer: an update. Acta Clin Belg. 2012 July-August; 67(4):270-5).

In some embodiments, a report summarizing the results of the analysis, i.e. the diagnosis of the subject, and any other information pertaining to the analysis could optionally be generated as part of the analysis (which may be interchangeably referred to herein as “providing” a report, “producing” a report, or “generating” a report). Examples of reports may include, but are not limited to, reports in paper (such as computer-generated printouts of test results) or equivalent formats and reports stored on computer readable medium (such as a CD, computer hard drive, or computer network server, etc.). Reports, particularly those stored on computer readable medium, can be part of a database (such as a database of patient records, which may be a “secure database” that has security features that limit access to the report, such as to allow only the patient and the patient's medical practitioners to view the report, for example). In addition to, or as an alternative to, generating a tangible report, reports can also be displayed on a computer screen (or the display of another electronic device or instrument). In some embodiments, the report indicates that the subject has prostate cancer when the level of the one or more biomarkers is increased as compared to a reference level. In some embodiments, the report indicates that the subject does not have prostate cancer when the level of the one or more biomarkers is decreased or remains unchanged as compared to a reference level.

A report can further be transmitted, communicated or reported (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party intended to view or possess the report. The act of ‘transmitting’ or ‘communicating’ a report can be by any means known in the art, based on the form of the report, and includes both oral and non-oral transmission. Furthermore, “transmitting” or “communicating” a report can include delivering a report (“pushing”) and/or retrieving (“pulling”) a report. For example, non-oral reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

In some embodiments, the subject diagnosed with prostate cancer using the methods disclosed herein is referred for treatment of prostate cancer. In some embodiments, the subject is a post-prostate cancer treatment subject and is referred for treatment of recurrence of prostate cancer. The subject may be treated using radiation therapy, surgery, hormone therapy, chemotherapy, vaccine treatment, cryosurgery, and/or bone directed treatment.

The present disclosure is further illustrated by the following Examples, which in no way should be construed as further limiting. The entire contents of all of the references (including literature references, issued patents, published patent applications, and co pending patent applications) cited throughout this application are hereby expressly incorporated by reference.

### Examples

### Materials and Methods

### Urine Collection and Processing

Urine was collected according to our institutional bioethical guidelines regarding discarded clinical material and stored as previously reported.²¹ Specimens were obtained in The Urology Clinic at the Brigham and Women's Hospital, Boston, Mass., before surgical or other therapeutic interventions and were collected in sterile containers as voided urine and immediately frozen at −20° C. Urine was tested for the presence of blood and leukocytes using Multistix 9 strips (Siemens Healthcare Diagnostics Inc., Tarrytown, N.Y.), and samples containing blood or leukocytes were excluded.²¹ None of the patients had clinical signs or documentation of prostatitis or UTI.

One hundred seventy-three (173) samples were analyzed in this study, including samples from patients diagnosed with organ-confined benign prostate hyperplasia (n=83) and prostate cancer (n=90). Specimens taken from patients with organ-confined cancers were obtained prior to surgical or other therapeutic intervention. The two groups were not significantly different with respect to race (% Caucasian: BPH 80%, PCa 74% P=0.11, chi-square test). Gleason scores of the prostate adenocarcinomas ranged from 5 to 9, with 85 out of the 90 (94%) prostate adenocarcinomas having Gleason scores of 5-7. In addition, the prostate cancer group included stages T1-T3. None of the patients were diagnosed with metastatic cancer at the time of sampling. Mean age was not significantly different between the PCa and BPH and PCa groups (63.3 vs. 66.1 years, P=0.15). Samples were analyzed in a double-blinded manner.

LC/LC/MS/MS Identification of Differentially Expressed Proteins by Isobaric Tagging with iTRAQ (Isobaric Tags for Relative and Absolute Quantitation)

iTRAQ is a state-of-the-art quantitative mass spectrometry approach to identifying and quantifying components of the proteome present in biological samples. Proteins were obtained from human urine by organic precipitation with methanol following our previously published method.²² Protein profiling was performed using the 8-plex iTRAQ (AB Sciex, Foster City, Calif.) labeling protocol and standard MudPIT methodology coupled with the 4800 MALDI TOF/TOF Plus instrument to perform the mass spectrometry as previously described.²² Protein Pilot 2.0.1 software with the Paragon algorithm²³ was used for peptide and protein identification and relative quantitation based on the iTRAQ labels.

### Immunoblot Analyses

All 173 urine samples (BPH=83, PCa=90) were individually concentrated using an UltraFree-4 centrifugal filter device with a molecular weight cut off of 5 kDa (Millipore, Bedford, Mass., USA) as previously reported.^{21,24} Protein expression was detected by immunoblot analyses using monospecific primary antibodies: apoD, β2M, pepsin A, uromodulin, ZAG, (Santa Cruz Biotechnology, Santa Cruz, Calif., USA) and MUC3 (Thermo Fisher Scientific Inc, Rockford, Ill., USA). Proteins were evaluated via quantitative densitometry and expressed in densitometric units (DU) (Supplemental Methods).

### Pathways and Interactive Network Systems Biology Analysis

Ingenuity Pathway Analysis (IPA 7.0) (http//www.ingenuity.com) was used to identify key interaction networks and pathways significantly enriched in BPH and PCa urine samples. Based on the differentially expressed proteins, a network composed of interactive proteins was built using the network building and growing utility in the Ingenuity Pathway Analysis (IPA) tool. Enriched pathways within this hierarchical network were ranked using the ratio of affected proteins and Fisher's exact test. To identify the key regulatory molecules within this integrated network, we used the density of maximum neighborhood component (DMNC) algorithm.²⁵

### Statistical Analysis

Univariate analysis included a comparison of median levels of the six proteins and PSA between PCa and BPH patients by the nonparametric Mann-Whitney U-test since these variables showed some skewness and lack of normality as tested by the Kolmogorov-Smirnov goodness-of-fit statistic. Receiver operating characteristic (ROC) curve analysis was applied to determine the area under the curve (AUC) as a measure of predictive accuracy and the Youden J-index was used to identify the optimal cutoff value for each biomarker.²⁶ Multivariate logistic regression analysis using backward selection with the likelihood ratio test to assess significance was applied to identify the independent predictive biomarkers of PCa and derive the probability of PCa based on combinations of these biomarkers (using the optimal cutoff value in densitometric units) and stratified by PSA level based on three clinical categories (0-4, 4.1-10, >10 ng/ml). AUC was also calculated for combinations of biomarkers with 95% confidence intervals to determine whether improved prediction was achieved by combining significant biomarkers together using multivariate modeling with the c index used to quantify the combined predictive accuracy.²⁷ ROC curves were compared by the DeLong test.²⁸ Two-tailed values of P<0.05 were considered statistically significant. Statistical analysis was performed using IBM SPSS Statistics (version 21.0, IBM, Armonk, N.Y.).

### Chemicals

Methanol, ethanol, and 10% formalin were purchased from Thermo Fisher Scientific Inc. (Waltham, Mass., USA). Ethanol, EDTA, 2-mercaptoethanol, bovine serum albumin (BSA), and Triton x-100 were purchased from Sigma (St. Louis, Mo., USA). Tween 20 and Bradford protein reagent were purchased from Bio-Rad (Hercules, Calif., USA).

### Immunoblot Analyses

Protein concentration of urine samples was determined by the Bradford method (Bio-Rad, Hercules, Calif., USA). Equal amounts of proteins from urine (30 μg/lane) were separated on NuPAGE 4-12% Bis-Tris gels (Invitrogen, Carlsbad, Calif., USA) and transferred to PVDF membranes (Millipore, Bedford, Mass., USA). Protein expression was determined by appropriate primary antibodies: apoD, β-2M, pepsin A, uromodulin, ZAG, (Santa Cruz Biotechnology, Santa Cruz, Calif., USA) and MUC3 (Thermo Fisher Scientific Inc, Rockford, Ill., USA) diluted to 1:1000 in 5% BSA blocking solution. Secondary anti-mouse and anti-rabbit antibodies 1:2000 (Thermo Fisher Scientific Inc, Rockford, Ill., USA) were diluted in 5% BSA. The expression of each protein was detected by the ECL Western Lightning (PerkinElmer Life and Analytical Sciences, Inc., Waltham, Mass.) as previously reported1,2 and bands of interest corresponding to target proteins (apoD, β2M, pepsin A, uromodulin, ZAG, and MUC3) were evaluated via quantitative densitometry (Un-Scan-It, Silk Scientific Inc., Orem, Utah).

LC/LC/MS/MS Identification of Differentially Expressed Proteins by Isobaric Tagging with iTRAQ

Briefly, ice-cold 100% methanol was added to ice-cold urine to a final concentration of 75%. After mixing by inversion several times the tubes were stored overnight at −20 C. Precipitated proteins were collected by centrifugation (16,000×g, 15 min at room temperature). Precipitated proteins were washed three times with ice-cold 100% methanol and five-minute spins were employed to recover the protein after each wash. Collected proteins from each sample were then briefly air-dried in a fume hood and resuspended in milliQ water to an approximate concentration of 1.0 mg/ml. Protein concentration was determined using the BioRad Protein Assay (BioRad, Hercules, Calif., USA). A standard curve was generated using bovine serum albumin (BSA) and all assays were performed in triplicate with the mean value used for final calculations. All samples were analyzed by Coomassie stained SDS-PAGE (10% Tris-HCl Ready Gel, BioRad, Hercules, Calif., USA) to gauge the relative distribution of proteins in each sample versus intact albumin. Ten micrograms of protein was loaded per well and the gel assay confirmed stained protein in each sample.

Equal amounts of total protein from the human urine samples were used in the 8-plex-iTRAQ relative quantitation protocol following the manufacturer's recommendations (AB Sciex, Foster City, Calif., USA). Four urine samples from BPH patients and four samples from prostate cancer patients were used in one 8-plex-iTRAQ experiment. One hundred micrograms from each individual urine was dried in a centrifugal speed vacuum set-up. Each dried sample was then resuspended in 20 μl of dissolution buffer (0.5 M triethylammonium bicarbonate (TEAB), pH 8.5) containing 0.1% SDS. Proteins were reduced by adding 2 μl of 50 mM tris-(2-carboxymethyl)phosphine (TCEP) and incubated at 55° C. for one hour. Reduced disulfide bonds were then blocked by adding 1 μl of 200 mM methyl methanesulfonate (MMTS) and incubating at room temperature for 10 minutes. Proteins were digested into peptides using overnight trypsin digestion at 37° C. (added in ratio of 1:10). All peptides were then reacted with the 8-plex iTRAQ labels. After confirmation of addition of each label to each digested sample all eight samples were then pooled into one tube. Peptides were identified using the MudPIT methodology. First, peptides were separated on a strong cation exchange (SCX) column (POROS HS/20, 4.6 mm×100 mm (Applied Biosystems, Foster City, Calif., USA) attached to an Agilent 1100/1200 HPLC. Buffer A was 10 mM KH2PO4, 25% (vol/vol) acetonitrile, pH 2.8 and Buffer B was Buffer A plus 1 M KCl. A total of ninety-six fractions were collected. Based on the chromatogram at UV 214 the eluted fractions were pooled back into thirty tubes that spanned the range of resolved peptides.

In the second dimension separation, each pooled SCX fraction was then run over a reverse phase column (Acclaim PepMap100 C18, 5 μm, 100 A, 500 μm ID×5 mm, Dionex) using microflow and then resolved with nanoflow LC on an Ultimate Plus system (LC Packings/Dionex) over a 15 cm C18 column (Acclaim PepMap100, 3 μm, 75 μm ID×15 cm, Dionex) and then printed to 4800 MALDI target plates with an attached printing robot (Probot, Dionex). The MALDI matrix CHCA (Sigma, St. Louis, Mo., USA) was made as a 5.0 mg/ml stock solution and mixed in automatically by the Probot just prior to printing the samples on to the target plates. Peptides were identified by MALDI-TOF/TOF mass spectrometry on a 4800 Plus instrument (AB Sciex, Foster City, Calif., USA). Protein Pilot 2.0.1 software was used for peptide and protein identification and relative quantitation based on the iTRAQ labels. Protein Pilot used the Paragon algorithm.3 The latest available versions of three human protein databases were separately searched using Protein Pilot 2.0.1; SwissProt (non-redundant, 18 053 proteins), TrEMBL (redundant, 55 942 proteins) and NCBI (redundant, 176 140 proteins).

### Results

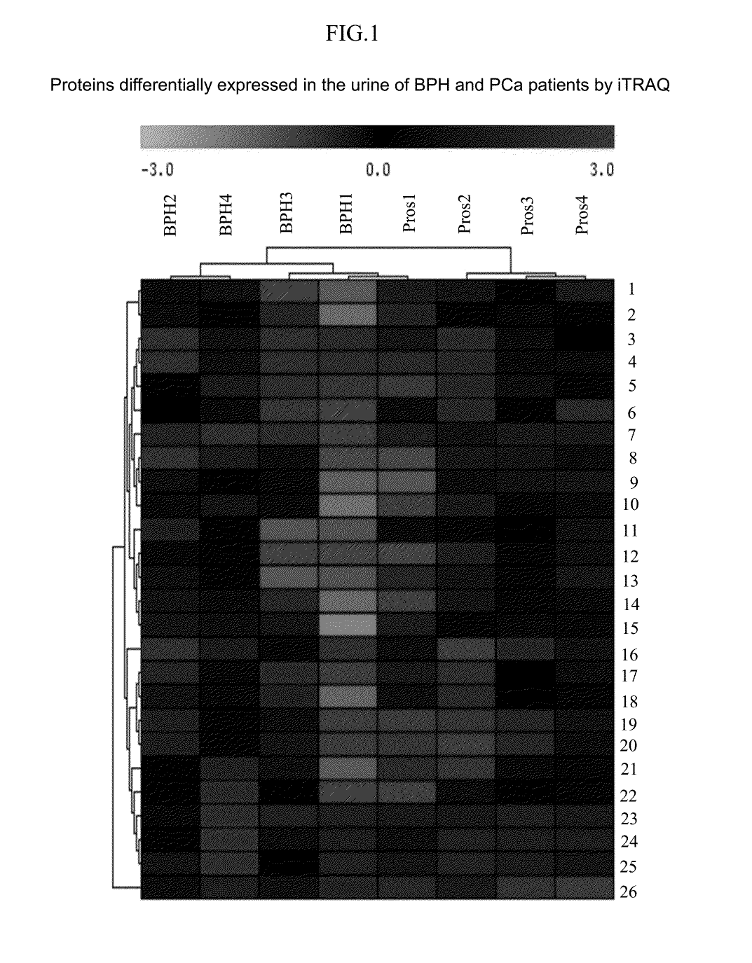

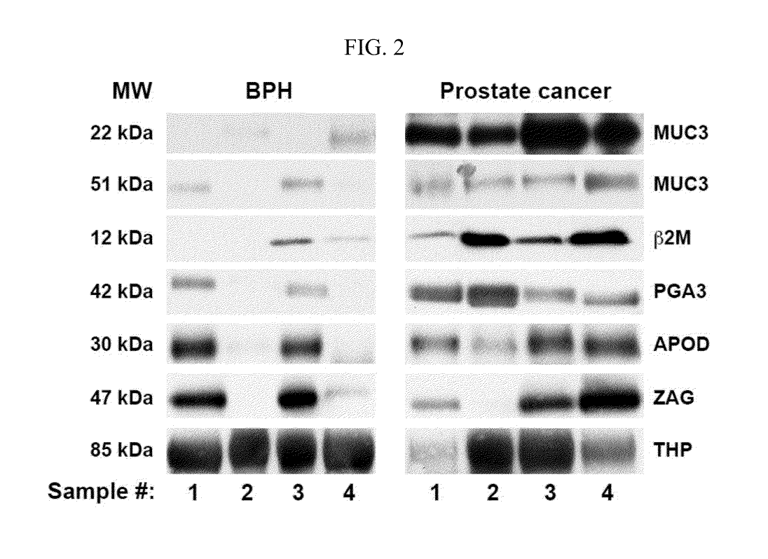

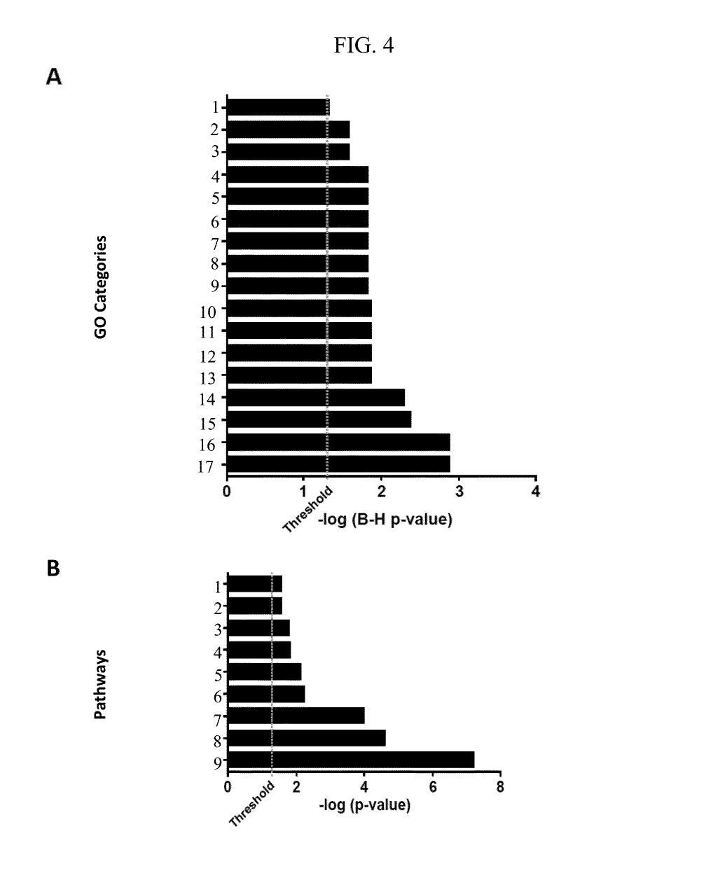

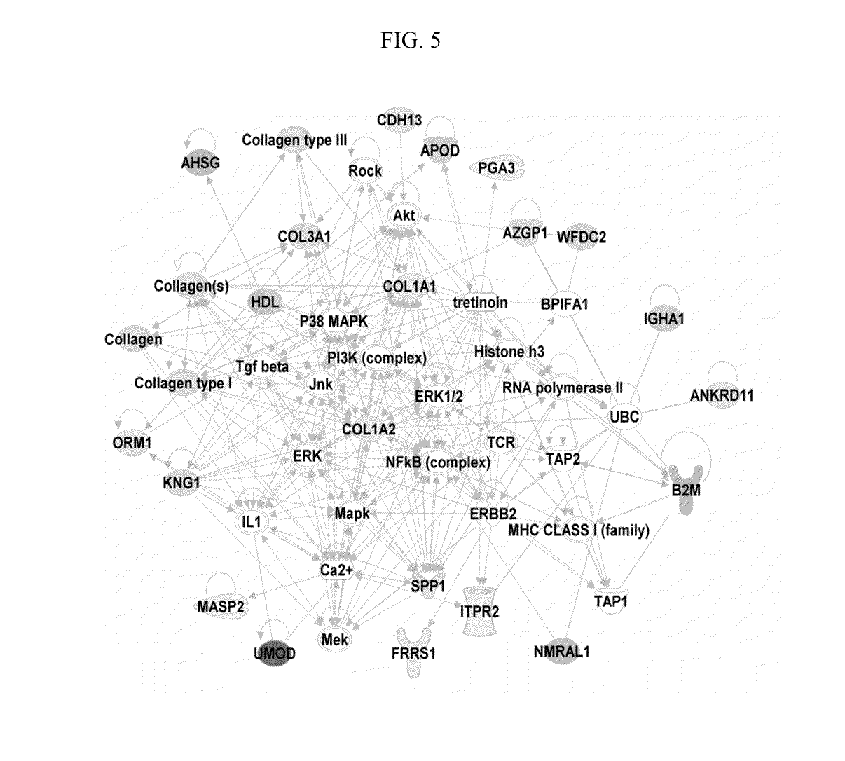

iTRAQ LC/LC/MS/MS was utilized to sensitively and accurately identify the urinary proteome of men with BPH vs. prostate cancer. This approach identified 25 proteins that were differentially expressed, at a significant level, with high confidence, in urines from men from each of the two groups of interest (PCa and BPH) (FIG. 1). Functional enrichment analysis and pathways enrichment analysis of these proteins using Ingenuity Pathway Analysis (IPA) tools was then performed to determine differentially expressed pathways and functions in prostate cancer as compared to BPH. These proteins represent a number of different functional categories including cell assembly and organization, cell signaling, cell morphology, carbohydrate metabolism, cellular growth and proliferation, lipid metabolism, androgen and estrogen metabolism, and DNA replication, recombination, and repair, among others (FIGS. 4A and 4B). Additionally, network analysis identified differences in many focus hubs (e.g., NFκB, ERK1/2, Collagen, TGFβ, PI3K, p38 MAPK) with high a degree of interactions (FIG. 5).

26 proteins were differentially expressed in the urine of patients diagnosed with BPH vs. PCa as originally identified by iTRAQ (FIG. 1). Given that enzyme-linked immunosorbent assays (ELISA) for these proteins were not available, nine proteins were tested based on the availability of antibodies, and then six proteins were validated: β-2-microglobulin (β2M), pepsinogen 3, group 1 (PGA3), intestinal mucin (MUC3), apolipoprotein D (APOD), alpha-2-glycoprotein 1, zinc (ZAG), and uromodulin (THP) (FIG. 2). These analyses provided validation that β2M, PGA3 and MUC3 were differentially expressed in the urine of patients diagnosed with PCa and BPH.

Univariate analysis was performed to compare BPH and PCa groups with respect to age, and each of the candidate urinary biomarkers (Table 1).

TABLE 1
Comparison of Urinary Proteins That Distinguish Between BPH and PCa
Cohorts

| | PCa | | BPH | | Area under the Curve | |
|---|---|---|---|---|---|---|
| Variable | (N = 90) | | (N = 83) | | AUC | P value |
| Age, years, mean | 63.3 ± 8.7 | | 66.1 ± 8.4 | | — | 0.15 |
| B-2 microglobulin | 143.4 | (44.5-289.8) | 30.3 | (4.2-194.6) | 0.658 | <0.001* |
| PGA3 | 198 | (32-329) | 106 | (8-263) | 0.623 | 0.006* |
| MUC3 25 kDa | 421 | (239-490) | 322 | (93-465) | 0.605 | 0.018* |
| MUC3 51 kDa | 33.0 | (6.3-104.9) | 18.7 | (3.2-68.4) | 0.583 | 0.06 |
| PIK3IP1 | 10.0 | (2.6-41.2) | 2.9 | (1.3-12.1) | 0.641 | 0.002* |
| APOD | 381 | (134-512) | 255 | (35-486) | 0.568 | 0.14 |
| Uromodulin-THP | 267 | (122-368) | 232 | (89-377) | 0.547 | 0.30 |
| ZAG | 384 | (144-529) | 351 | (89-523) | 0.522 | 0.64 |

Biomarker data are median and interquartile range.
*Statistically significant

These comparisons, based on continuous data, revealed significant elevations in β2M (P<0.001), PGA3 (P=0.006), and MUC3 25 kDa (P=0.018). The optimal cut-off values was then identified using ROC analysis with the Youden index for each of the three significant urinary biomarkers, and the analysis indicated ≧40 DU for β2M, ≧190 DU for PGA3, and ≧185 DU for MUC3. Multivariate logistic regression modeling was then conducted using the chosen cut-off values for each of the three significant biomarkers in the univariate analysis as well as PSA (using three clinically defined categories: 0-4 ng/mL, 4.1-10 ng/mL, >10 ng/mL).

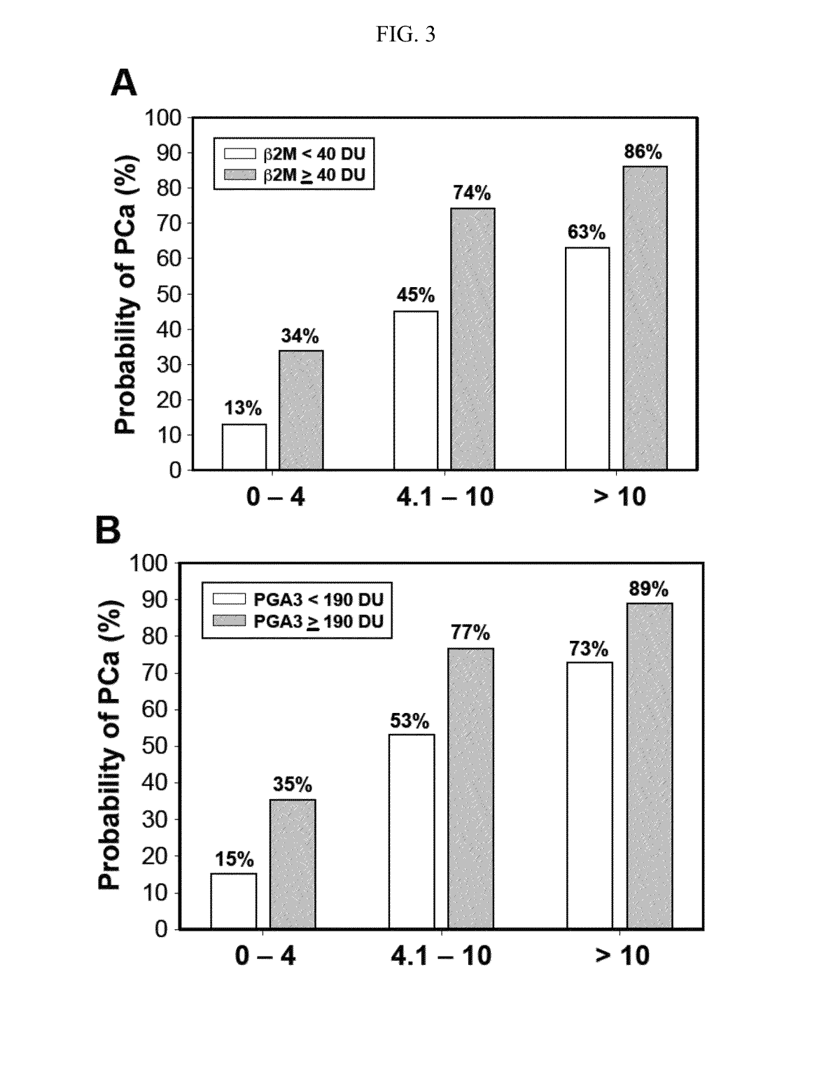

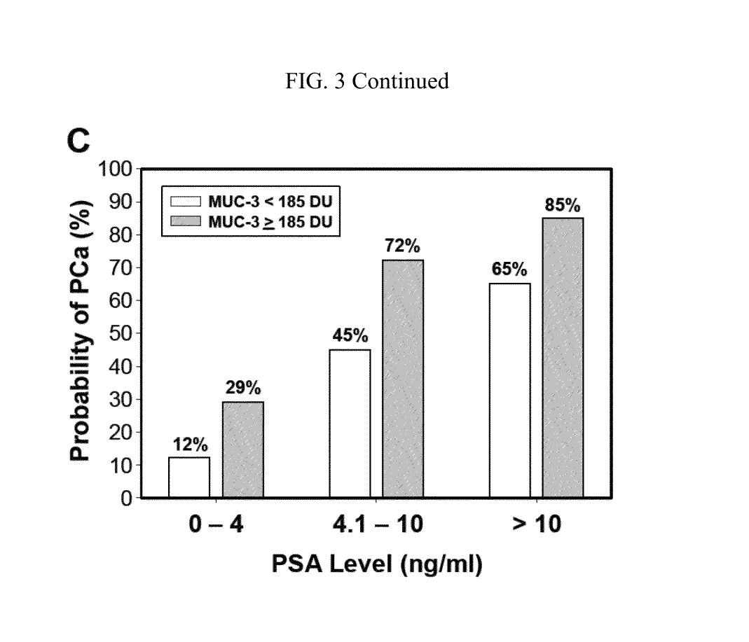

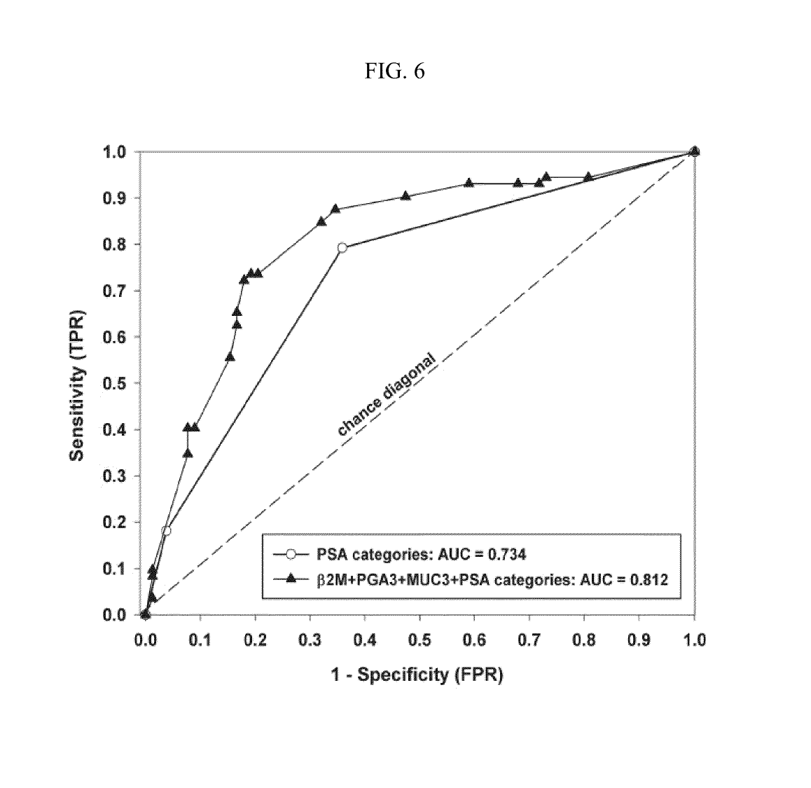

The probability of PCa was determined using multivariate logistic regression modeling according to the each of the urinary biomarkers, β2M, PGA3, MUC3, as well as PSA in the predictive model. Here, having determined the optimal cut-off value for each biomarker based on the Youden J-index in receiver operating characteristic (ROC) curve analysis, the binary predictors (i.e., above and below each cut-off) stratified according to clinically relevant categories of PSA were used. Thus, the probabilities shown in each panel of FIG. 3 are based on two levels of the biomarker for each of three PSA categories (0-4, 4.1-10, >10 ng/mL). The multivariate modeling strategy uses the Newton-Raphson algorithm in maximum likelihood estimation (MLE) to derive the probability of PCa based on combinations of the biomarkers within each PSA category. This figure illustrates that the estimated probability of PCa within each PSA category is significantly higher in patients who are above the cut-off value for the biomarker and that the probability of PCa is higher with increasing PSA. Receiver-operating characteristic analyses revealed that if the levels of β2M are less than 40 DU, if the PGA3 levels are less than 195 DU and the MUC3 levels are less than 185 DU, the predictive accuracy is improved to 45%, 53% and 45%, respectively. However, when the DU levels are equal to or higher than 40 DU of β2M, 195 DU of PGA3 and 185 DU of MUC3, the diagnostic accuracy is remarkably improved to 74%, 77% and 72%, respectively (FIG. 3). In addition, the receiver operating characteristic curve (ROC) clearly shows a steeper curve for the three urinary biomarkers (MUC3, PGA3, β2M) as well as for the three biomarkers plus PSA categories (FIG. 6).

To determine the predictive accuracy of each of the significant independent multivariate biomarkers based on the optimal cutoff values and PSA based on the three categories, ROC analysis was used to assess the AUC for single biomarkers and the combination of three with and without PSA (Table 2).

TABLE 2

| Biomarker | AUC | 95% CI | P value |
|---|---|---|---|
| β2M ≧40 DU | 0.668 | 0.628-0.748 | <0.001* |
| PGA3 ≧190 DU | 0.625 | 0.547-0.710 | 0.008* |
| MUC3 ≧185 DU | 0.618 | 0.532-0.700 | 0.009* |
| PSA categories, ng/mL (0-4.0, 4.1-10, | 0.734 | 0.653-0.814 | 0.007* |
| >10) | | | |
| β2M + PGA3 + MUC3 | 0.710 | 0.631-0.788 | <0.001* |
| β2M + PGA3 + MUC3 + PSA categories | 0.812 | 0.740-0.885 | <0.001* |

Single biomarkers had AUC values ranging from 0.618 for MUC3 25 kDa (P=0.009) to 0.668 for β2M (P<0.001); the combination of β2M, PGA3 and MUC3 25 kDa increased the AUC to 0.710 (95% CI: 0.631-0.788, P<0.001). Predictive accuracy was 0.734 based on PSA categories alone and significantly increased to 0.812 for the three biomarkers with PSA categories (P=0.004, Delong test for comparing ROC curves). False positive (FPR) and false negative rates (FNR) are highly relevant in clinical practice and we have evaluated the FPR and FNR for each of the three significant multivariate predictive biomarkers in differentiating between BPH and PCa. It is clear that compared to each of the three urinary biomarkers alone, the panel of three biomarkers combined provide much lower FPRs and FNRs based on all patients in the study population. The combination of three biomarkers together (β2M, PGA3, MUC3) shows an FPR of 30% and a range of FNRs from 0% to 8%, which is considered clinically acceptable.

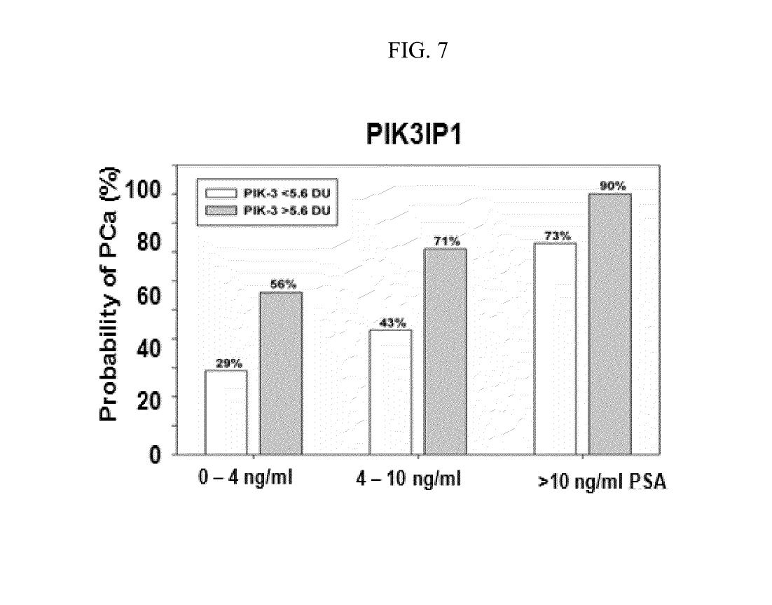

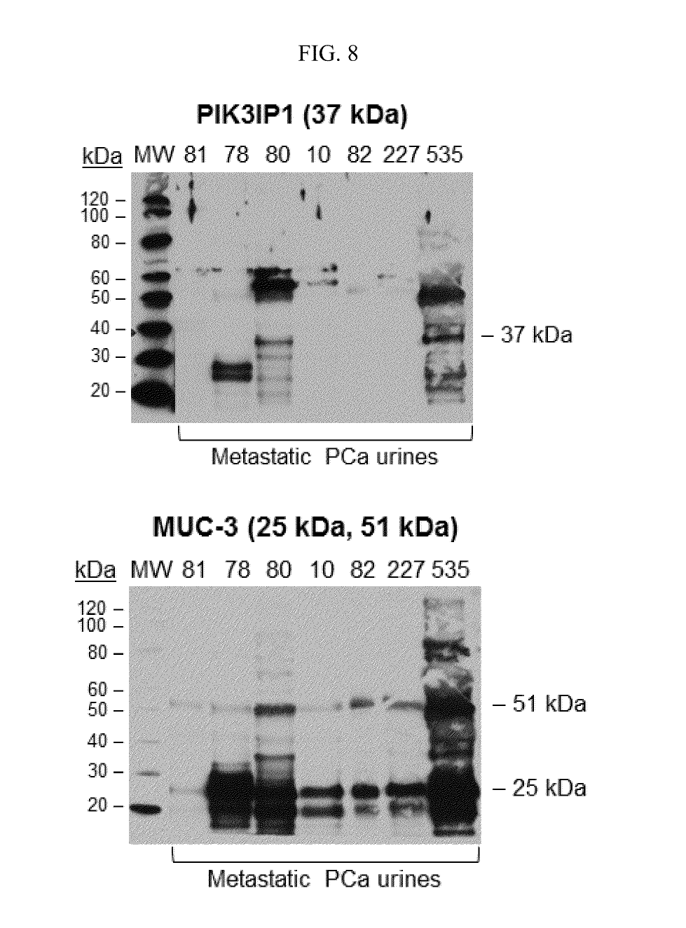

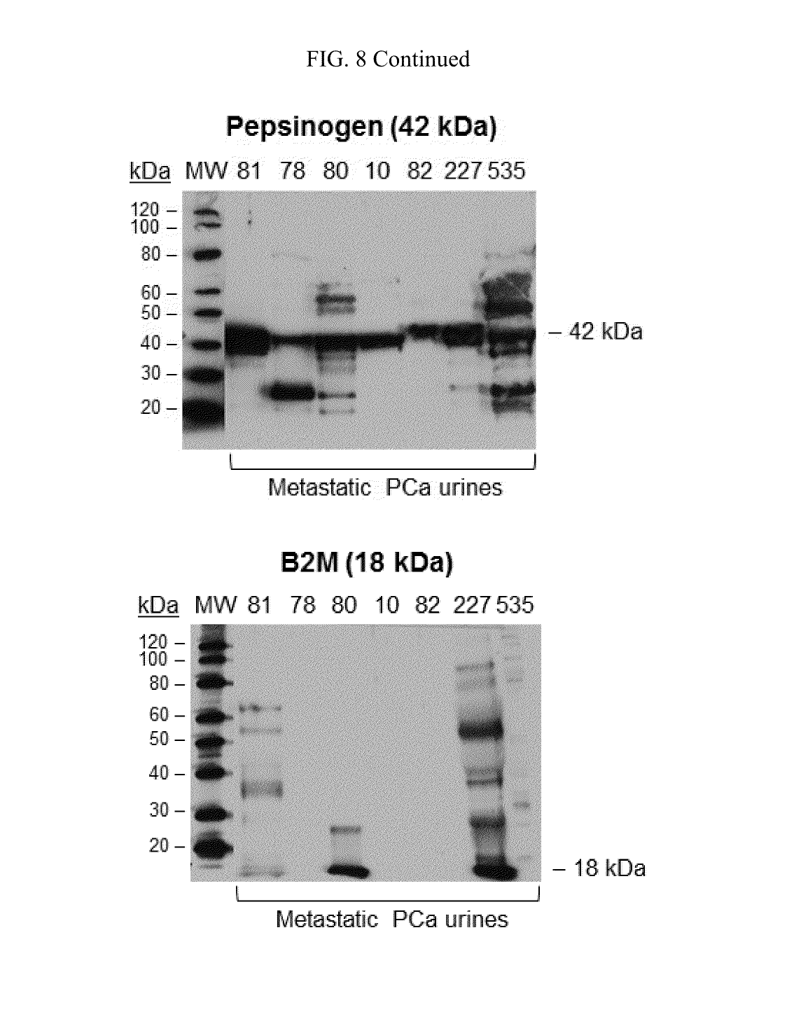

Univariate analysis revealed that PIK3IP1 is significantly elevated (Table 3) in PCa patients (P=0.002). Diagnostic accuracy for each biomarker and their combination was based on optimal cut off values from ROC (receiver operating characteristic) analysis (Table 4). Moreover, multivariate logistic regression based on optimal PIK3IP1 cut off values was used to derive the probability of PCa for PIK3IP1 stratified by the clinically defined categories of PSA. Probability of PCa within each of the three PSA categories is shown based on whether or not the patient's PIK3IP1 is >5.6 DU or <5.6 DU, and these probabilities increase with higher PSA (FIG. 7).

TABLE 3
Comparison of Urinary Proteins Between PrCa and BPH Cohorts

| | PrCa | | BPH | | | |
|---|---|---|---|---|---|---|
| Biomarker | (N = 90) | | (N = 83) | | AUC | P value |
| Age, years, mean ± SD | 63.3 ± 8.7 | | 66.1 ± 8.4 | | — | 0.15 |
| Mucin-3 25 kDa | 421 | (239-490) | 322 | (93-465) | 0.605 | 0.018* |
| Mucin-3 51 kDa | 33.0 | (6.3-104.9) | 18.7 | (3.2-68.4) | 0.583 | 0.06 |
| B2M | 143.4 | (44.5-289.8) | 30.3 | (4.2-194.6) | 0.658 | <0.001* |
| Pepsinogen | 198 | (32-329) | 106 | (8-263) | 0.623 | 0.006* |
| APOD | 381 | (134-512) | 255 | (35-486) | 0.568 | 0.14 |
| THP | 267 | (122-368) | 232 | (89-377) | 0.547 | 0.30 |
| ZAG | 384 | (144-529) | 351 | (89-523) | 0.522 | 0.64 |
| PIK3IP1 | 10.0 | (2.6-41.2) | 2.9 | (1.3-12.1) | 0.641 | 0.002* |

Biomarker data are median (interquartile range).
*Statistically significant.

TABLE 4
Diagnostic Accuracy in Predicting PCa vs. BOH Based on Optimal Cutoff
Values from ROC Analysis

| Biomarker | AUC | 95% CI | P value |
|---|---|---|---|
| β2M ≧40 DU | 0.668 | 0.628-0.748 | <0.001 |
| PGA3 ≧190 DU | 0.625 | 0.547-0.710 | 0.008 |
| MUC3 ≧185 DU | 0.617 | 0.535-0.701 | 0.009 |
| PIK3IP1 ≧5.6 DU | 0.648 | 0.567-0.731 | 0.001 |
| β2M + PGA3 + MUC3 | 0.709 | 0.631-0.787 | <0.001 |
| β2M + PGA3 + MUC3 + PIK3IP1 | 0.748 | 0.675-0.820 | <0.001 |

TABLE 5
Comparison of Urinary Proteins Between Normal and PrCa samples.
Comparison of Urinary Proteins Between Normal and PrCa samples

| Protein | Normal (n = 41) | PrCa (n = 90) | P-value |
|---|---|---|---|
| MUC-3 25 kDa | 451 | 418 | 0.44 |
| MUC-3 51 kDa | 38 | 32 | 0.82 |
| PIK-3IP1 | 0.98 | 9 | 0.013* |
| B-2 microglobulin | 140 | 128 | 0.706 |
| Pepsinogen | 340 | 171 | 0.0009* |

### Discussion

This study was designed to evaluate new markers in a patient population that would undergo screening in common clinical practice. The U.S. Preventive Task Force strongly rejected the utility of PSA screening for prostate cancer and this study was designed to determine if new urinary markers would be more informative in discriminating between BPH and prostate cancer which is the problem facing clinicians. The clinical challenge is differentiating BPH from prostate cancer.

In recent years, urinary biomarkers have emerged as an attractive option for the noninvasive detection of PCa.^{21,29-31} Given the complexity of this disease, it is now widely appreciated that a single marker may not necessarily reflect the multifactorial nature of BPH or PCa.²⁹ A panel, rather than any individual biomarker, will have a higher likelihood to more accurately distinguish between BPH and early stages of PCa in conjunction with clinicopathological parameters. In contrast, the newly identified biomarkers, such as β2M, PGA3, MUC3 and PIK3IP1, effectively discriminated BPH from early PCa.

A protein found to be significantly elevated in urine of PCa patients was mucin 3 (MUC3), a member of the membrane-associated mucins, which may be shed from the cell surface via activation of membrane-associated metalloproteinases.³²⁻³⁴ Previous studies showed a correlation between elevated MUC3 expression and esophageal,³⁵ gastric,³⁶ breast,³⁷ and colon cancer.³⁸ In this study, MUC3 was able to differentiate between BPH and early PCa. In addition, the ability of MUC3 to discriminate between BPH and early PCa was strengthened when MUC3 was multiplexed with clinically defined categories of PSA, making it a prospective biomarker for differentiating BPH from early PCa.

Pepsinogen 3, group 1 (PGA3) was also found to be elevated in the urine of PCa patients but not in BPH. PGA3 is synthesized and secreted by the gastric chief cells of the human stomach before being converted into the proteolytic enzyme pepsin A, an upstream step in the digestive process.³⁹ Low levels of PGA in serum⁴⁰ as well as decreased or lost expression of PGA in gastric tissue and cancer cell lines were previously reported.⁴¹ In contrast, a recent study demonstrated increased mRNA levels of PGA in seven colorectal cancer cell lines.⁴² Interestingly, this study is the first to report that PGA3 can be used to distinguish between patients with BPH or with early-stage PCa.

β2M, a component of the major histocompatibility complex class I (MHC I), was the third protein identified via iTRAQ and validated by immunoblot analysis. Increased expression of β2M has been previously associated with breast,⁴³ renal,⁴⁴ lung,⁴⁵ colon,⁴⁶ and hematologic malignancies.⁴⁷ β2M levels were also significantly elevated in urine⁴⁸ and in serum⁴⁹ of prostate cancer patients compared to healthy subjects. This is the first study demonstrating that β2M effectively discriminates between BPH and early stages of PCa.

Taken together, these new biomarkers such as β2M, PGA3, PIK3IP1 and MUC3 are able to independently discriminate between BPH and early stages of PCa. In addition, when these markers are multiplexed, the accuracy in differentiating between BPH and early PCa is further increased. Importantly, this small panel of biomarkers, when multiplexed with clinically defined categories of PSA, effectively distinguishes BPH from early PCa with high sensitivity and specificity. In summary, noninvasive urine tests utilizing the biomarkers described herein, such as β2M, PGA3, PIK3IP1 and MUC3 in combination with clinically defined categories of PSA, have the potential to resolve the diagnostic dilemma of effectively discriminating between BPH and early PCa in the clinic.

### REFERENCES

1. Jemal A, Center M M, DeSantis C et al: Global patterns of cancer incidence and mortality rates and trends. Cancer Epidemiol Biomarkers Prev 2010; 19: 1893.
2. Siegel R, Naishadham D and Jemal A: Cancer statistics, 2013. CA Cancer J Clin 2013; 63: 11.
3. Gooren L: Androgen deficiency in the aging male: benefits and risks of androgen supplementation. J Steroid Biochem Mol Biol 2003; 85: 349.
4. Bosch J L, Hop W C, Kirkels W J et al: Natural history of benign prostatic hyperplasia: appropriate case definition and estimation of its prevalence in the community. Urology 1995; 46: 34.
5. Guess H A: Epidemiology and natural history of benign prostatic hyperplasia. The Urol Clin North Am 1995; 22: 247.
6. Abrams P: New words for old: lower urinary tract symptoms for “prostatism”. BMJ 1994; 308: 929.
7. Eckhardt M D, van Venrooij G E, van Melick H H et al: Prevalence and bothersomeness of lower urinary tract symptoms in benign prostatic hyperplasia and their impact on well-being. J Urol 2001; 166: 563.
8. Siegel R, DeSantis C, Virgo K et al: Cancer treatment and survivorship statistics, 2012. CA Cancer J Clin 2012; 62: 220.
9. Pandha H, Sorensen K D, Orntoft T F et al: Urinary engrailed-2 (EN2) levels predict tumour volume in men undergoing radical prostatectomy for prostate cancer. BJU Int 2012; 110: E287.
10. Mazzucchelli R, Colanzi P, Pomante R et al: Prostate tissue and serum markers. Adv Clin Path 2000; 4: 111.
11. Thompson I M, Pauler D K, Goodman P J et al: Prevalence of prostate cancer among men with a prostate-specific antigen level < or =4.0 ng per milliliter. N Engl J Med 2004; 350: 2239.
12. Catalona W J, Richie J P, Ahmann F R et al: Comparison of digital rectal examination and serum prostate specific antigen in the early detection of prostate cancer: results of a multicenter clinical trial of 6630 men. J Urol 1994; 151: 1283.
13. Coley C M, Barry M J, Fleming C et al: Early detection of prostate cancer. Part 1: Prior probability and effectiveness of tests. The American College of Physicians. Ann Intern Med 1997; 126: 394.
14. Andriole G L, Crawford E D, Grubb R L, 3rd et al: Mortality results from a randomized prostate-cancer screening trial. New Eng J Med 2009; 360: 1310.
15. Basch E, Oliver T K, Vickers A et al: Screening for prostate cancer with prostate-specific antigen testing: American Society of Clinical Oncology Provisional Clinical Opinion. J Clin Oncol 2012; 30: 3020.
16. Greene K L, Albertsen P C, Babaian R J et al: Prostate specific antigen best practice statement: 2009 update. J Urol 2009; 182: 2232.
17. U.S. Preventive Services Task Force recommendation statement. Screening for Prostate Cancer (http://www.uspreventiveservicestaskforce.org/prostatecancer screening.htm) 2012.
18. Yang C C, Sun S S, Lin C Y et al: Differentiation of prostate cancer and benign prostatic hyperplasia: the clinical value of 201T1 SPECT a pilot study. Ann Nuc Med 2003; 17: 521.
19. Pal R P, Maitra N U, Mellon J K et al: Defining prostate cancer risk before prostate biopsy. Urol Oncol 2013; 31:1408.
20. Jamaspishvili T, Kral M, Khomeriki I et al: Urine markers in monitoring for prostate cancer. Prostate Cancer Prostatic Dis 2010; 13: 12.
21. Moses M A, Wiederschain D, Loughlin K R et al: Increased incidence of matrix metalloproteinases in urine of cancer patients. Cancer Res 1998; 58: 1395.
22. Afkarian M, Bhasin M, Dillon S T et al: Optimizing a proteomics platform for urine biomarker discovery. Mol Cell Proteomics 2010; 9: 2195.
23. Shilov I V, Seymour S L, Patel A A et al: The Paragon Algorithm, a next generation search engine that uses sequence temperature values and feature probabilities to identify peptides from tandem mass spectra. Mol Cell Proteomics 2007; 6: 1638.
24. Roy R, Louis G, Loughlin K R et al: Tumor-specific urinary matrix metalloproteinase fingerprinting: identification of high molecular weight urinary matrix metalloproteinase species. Clin Cancer Res 2008; 14: 6610.
25. Lin C Y, Chin C H, Wu H H et al: Hubba: hub objects analyzer a framework of interactome hubs identification for network biology. Nucleic Acids Res 2008; 36: W438.
26. Faraggi D and Reiser B: Estimation of the area under the ROC curve. Stat Med 2002; 21: 3093.
27. Zurakowski D, Johnson V M and Lee E Y: Biostatistics in clinical decision making for cardiothoracic radiologists. J Thorac Imaging 2013; 28: 368.
28. DeLong E R, Delong D M and Clarke-Pearson D L: Comparing the areas under two or more correlated receiver operating characteristic curves: a nonparametric approach. Biometrics 1988; 44: 837.
29. Rigau M, Olivan M, Garcia M et al: The present and future of prostate cancer urine biomarkers. Int J Mol Sci 2013; 14: 12620.
30. Roy R, Coticchia C C, Yang J et al: Biomarkers in Cancer Biology. In: Biomarkers of Toxicity: A New Era in Medicine. Edited by Vidaya V and Bonventre J. New York, N.Y.: John Wiley and Sons, pp. 355-380, 2010.
31. Haj-Ahmad T A, Abdalla M A and Haj-Ahmad Y: Potential Urinary Protein Biomarker Candidates for the Accurate Detection of Prostate Cancer among Benign Prostatic Hyperplasia Patients. J Cancer 2014; 5: 103.
32. Hollingsworth M A and Swanson B J: Mucins in cancer: protection and control of the cell surface. Nat Rev Cancer 2004; 4: 45.
33. Thathiah A, Blobel C P and Carson D D: Tumor necrosis factor-alpha converting enzyme/ADAM 17 mediates MUC1 shedding. J Biol Chem 2003; 278: 3386.
34. Thathiah A and Carson D D: MT1-MMP mediates MUC1 shedding independent of TACE/ADAM17. Biochem J 2004; 382: 363.
35. Arul G S, Moorghen M, Myerscough N et al: Mucin gene expression in Barrett's oesophagus: an in situ hybridisation and immunohistochemical study. Gut 2000; 47: 753.
36. Wang R Q and Fang D C: Alterations of MUC1 and MUC3 expression in gastric carcinoma: relevance to patient clinicopathological features. J Clin Pathol 2003; 56: 378.
37. Rakha E A, Boyce R W, Abd El-Rehim D et al: Expression of mucins (MUC1, MUC2, MUC3, MUC4, MUC5AC and MUC6) and their prognostic significance in human breast cancer. Mod Pathol 2005; 18: 1295.
38. Duncan T J, Watson N F, Al-Attar A H et al: The role of MUC1 and MUC3 in the biology and prognosis of colorectal cancer. World J Surg Oncol 2007; 5: 31.
39. Gritti I, Banfi G and Roi G S: Pepsinogens: physiology, pharmacology pathophysiology and exercise. Pharmacol Res 2000; 41: 265.
40. Westerveld B D, Pals G, Lamers C B et al: Clinical significance of pepsinogen A isozymogens, serum pepsinogen A and C levels, and serum gastrin levels. Cancer 1987; 59: 952.
41. Ichinose M, Miki K, Wong R N et al: Methylation and expression of human pepsinogen genes in normal tissues and their alteration in stomach cancer. Jpn J Cancer Res 1991; 82: 686.
42. Tani Y, Akiyama Y, Fukamachi H et al: Transcription factor SOX2 up-regulates stomach-specific pepsinogen A gene expression. J Cancer Res Clin Oncol 2007; 133: 263.
43. Teasdale C, Mander A M, Fifield R et al: Serum beta2-microglobulin in controls and cancer patients. Clin Chim Acta 1977; 78: 135.
44. Hemmingsen L and Skaarup P: Urinary excretion of ten plasma proteins in patients with renal carcinoma. Scan J Urol Nephrol 1977; 11: 41.
45. Shuster J, Gold P and Poulik M D: beta 2-microglogulin levels in cancerous and other disease states. Clin Chim Acta 1976; 67: 307.
46. Ward D G, Nyangoma S, Joy H et al: Proteomic profiling of urine for the detection of colon cancer. Proteome Sci 2008; 6: 19.
47. Yang J, Qian J, Wezeman M et al: Targeting beta2-microglobulin for induction of tumor apoptosis in human hematological malignancies. Cancer Cell 2006; 10: 295.
48. Abdul M and Hoosein N: Changes in beta-2 microglobulin expression in prostate cancer. Urol Oncol 2000; 5: 168.
49. Gross M, Top I, Laux I et al: Beta-2-microglobulin is an androgen-regulated secreted protein elevated in serum of patients with advanced prostate cancer. Clin Cancer Res 2007; 13: 1979.
50. Moses M A, Wiederschain D, Loughlin K R et al: Increased incidence of matrix metalloproteinases in urine of cancer patients. Cancer Res 1998; 58: 1395.
51. Roy R, Louis G, Loughlin K R et al: Tumor-specific urinary matrix metalloproteinase fingerprinting: identification of high molecular weight urinary matrix metalloproteinase species. Clin Cancer Res 2008; 14: 6610.
52. Shilov I V, Seymour S L, Patel A A et al: The Paragon Algorithm, a next generation search engine that uses sequence temperature values and feature probabilities to identify peptides from tandem mass spectra. Mol Cell Proteomics 2007; 6: 1638.

The foregoing written specification is considered to be sufficient to enable one skilled in the art to practice the disclosure. The present disclosure is not to be limited in scope by examples provided, since the examples are intended as a single illustration of one or more aspects of the disclosure and other functionally equivalent embodiments are within the scope of the disclosure.

Various modifications of the disclosure in addition to those shown and described herein will become apparent to those skilled in the art from the foregoing description and fall within the scope of the appended claims. The advantages and objects of the disclosure are not necessarily encompassed by each embodiment of the disclosure.

What is claimed is:

1. A method for distinguishing prostate cancer from benign prostatic hyperplasia (BPH) in a subject, the method comprising:
determining in a sample of the subject a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and
SNC66 protein, and
correlating the level of the one or more biomarkers to a reference level to facilitate diagnosis of prostate cancer or BPH.

2. The method of claim 1, further comprising providing a report indicating that the subject has prostate cancer when the level of the one or more biomarkers is increased as compared to a reference level.

3. The method of claim 1, further comprising providing a report indicating that the subject does not have prostate cancer when the level of the one or more biomarkers is decreased or remains unchanged as compared to a reference level.

4. The method of claim 1, wherein the reference level is the level of the one or more biomarkers in a control subject who does not have prostate cancer.

5. The method of claim 4, wherein the control subject has BPH.

6. The method of claim 1, wherein the biomarkers comprise MUC3, PGA3, β2M and PIK3IP1.

7. The method of claim 1, wherein the biomarker is not β2M.

8. The method of claim 1, wherein the sample is selected from the group consisting of blood, serum, urine, prostatic fluid, seminal fluid, semen, and prostate tissue.

9-14. (canceled)

15. A method for distinguishing prostate cancer from benign prostatic hyperplasia (BPH) in a subject, the method comprising:
performing an assay to determine a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein in a biological sample obtained of the subject, and
providing a report indicating that the subject has prostate cancer when the level of the one or more biomarkers is increased as compared to a reference level or providing a report indicating that the subject does not have prostate cancer when the level of the one or more biomarkers is decreased or remains unchanged as compared to the reference level.

16. A method for diagnosing prostate cancer in a subject, the method comprising:
determining in a biological sample of the subject a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and
SNC66 protein,
wherein an increased level of the one or more biomarkers as compared to a reference level is indicative that the subject has prostate cancer, and wherein a decreased level or an unchanged level of the one or more biomarkers as compared to a reference level is indicative that the subject does not have prostate cancer.

17. The method of claim 16, further comprising providing a report indicating that the subject has prostate cancer when the level of the one or more biomarkers is increased as compared to a reference level.

18. The method of claim 16, further comprising providing a report indicating that the subject does not have prostate cancer when the level of the one or more biomarkers is decreased or remains unchanged as compared to a reference level.

19-20. (canceled)

21. The method of claim 16, wherein the biomarkers comprise MUC3, PGA3, β2M and PIK3IP1.

22. The method of claim 16, wherein the biomarker is not β2M.

23. The method of claim 16, wherein the sample is selected from the group consisting of blood, serum, urine, prostatic fluid, seminal fluid, semen, and prostate tissue.

24-29. (canceled)

30. A method for monitoring prostate cancer recurrence in a post-prostate cancer treatment subject, the method comprising
determining a level of one or more biomarkers selected from the group consisting of: mucin 3 (MUC3); pepsinogen 3 preproprotein (PGA3); β-2-microglobulin (β2M); PIK3IP1; uromodulin; prion protein; apolipoprotein D; WAP four-disulfide core domain protein 2; kininogen 1 variant; collagen alpha-1(III) chain; osteopontin-c (OPN-c); epidermal growth factor (beta-urogastrone); unnamed protein product (GI 158261423); cadherin-13 isoform 1 preproprotein; collagen alpha 1 chain precursor variant; ankyrin repeat domain-containing protein 11; pro-alpha 2(I) collagen; sulfatase 2 isoform b precursor; MASP-2 protein; Inositol 1,4,5-triphosphate receptor, type 2, isoform CRA_b; unnamed protein product (GI 47077082); alpha-1-acid glycoprotein 1 precursor; Zinc-alpha-2-glycoprotein precursor (ZAG); HSCARG protein, isoform CRA_b; alpha2-HS glycoprotein; and SNC66 protein in a first biological sample of a post-prostate cancer treatment subject,
determining a level of the one or more biomarkers in a second biological sample of the post-prostate cancer treatment subject,
comparing the level of the one or more biomarkers in the first and second biological samples, and
referring the post-prostate cancer treatment subject for treatment of recurrence of prostate cancer when the level of the one or more biomarkers in the second biological sample is increased as compared to the level of the one or more biomarkers in the first biological sample.

31. The method of claim 30, further comprising providing a report indicating that the subject has recurrence of prostate cancer when the level of the one or more biomarkers in the second biological sample is greater than the level of the one or more biomarkers in the first biological sample.

32. The method of claim 30, further comprising providing a report indicating that the subject does not have recurrence of prostate cancer when the level of the one or more biomarkers in the second biological sample is decreased or remains unchanged as compared to the level of the one or more biomarkers in the first biological sample.

33. The method of claim 30, wherein the biomarkers comprise MUC3, PGA3, β2M and PIK3IP1.

34. (canceled)

35. The method of claim 30, wherein the sample is selected from the group consisting of blood, serum, urine, prostatic fluid, seminal fluid, semen, and prostate tissue.

36-41. (canceled)
